(19) United States
(12) Patent Application Publication
(10) Pub. No.: US 2022/0054532 A1
(43) Pub. Date: Feb. 24, 2022

(54) VETERINARY COMPOSITIONS AND METHODS OF USE THEREFOR

(71) Applicants: Colleen Smith, Chattanooga, TN (US); Diana Drumm, San Diego, CA (US)

(72) Inventors: Colleen Smith, Chattanooga, TN (US); Diana Drumm, San Diego, CA (US)

(21) Appl. No.: 17/435,549

(22) PCT Filed: Mar. 4, 2020
(86) PCT No.: PCT/US2020/020959

§ 371 Date: Sep. 1, 2021

Related U.S. Application Data
(60) Provisional application No. 62/813,832, filed on Mar. 5, 2019.
(60) Provisional application No. 62/813,840, filed on Mar. 5, 2019.

(51) Int. Cl. A61K 31/728; A61K 31/05; A61K 45/06; A61P 29/00; A61P 25/28; A61P 25/22; A61P 25/08; A61P 9/00; A61P 17/04

(52) U.S. Cl. CPC A61K 31/728; A61K 31/05; A61K 45/06; A61P 29/00; A61P 17/04; A61P 25/22; A61P 25/08; A61P 9/00; A61P 25/28

(57) ABSTRACT

Veterinary compositions comprising hyaluronic acid with a molecular weight of at least 800,000 Daltons and cannabidiol, veterinary compositions comprising hyaluronic acid with a molecular weight of at least 800,000 Daltons and cannabidiol Complex, and methods for treating ailments and/or symptoms of said ailments in non-human subjects in need thereof by administering an effective amount of such compositions.

### FIELD OF THE INVENTION

The invention relates generally to the field of veterinary compositions and methods for using said compositions to treat ailments and/or symptoms of said ailments. The invention relates to compositions comprising hyaluronic acid (“HA”) with a molecular weight of at least 800,000 Daltons (“high molecular weight HA” or “HMW HA”) and cannabidiol (“CBD”), to compositions comprising HMW HA and CBD Complex, and to methods for treating ailments and/or symptoms of said ailments by administering an effective amount of such compositions to non-human subjects in need thereof.

### BACKGROUND OF THE INVENTION

The global animal health market size was valued at $44.74 billion in 2018. The American Pet Products Association (“APPA”) reports that the total United States pet industry expenditures for 2017 was $69.51 billion and was estimated at $72.13 billion for 2018. In 2017, $17.07 billion was spent on veterinary care and $15.11 billion on supplies and over-the-counter medicine. The veterinary diagnostic market is predicted to reach $6.71 billion by 2021. According to that APPA's National Pet Owners Survey, 68% of households in the United States own a pet, i.e., 84.6 million homes, which is up from 56% in 1988.

Bob Vetere, the President and CEO of the APPA, noted that “[t]he industry is moving beyond simply the humanization of pets to where they are now widely accepted as part of the pet owner's lifestyle. No matter the primary cause of increased spending on pets, be it enhanced nutrition, better healthcare, increased pampering, or greater technology, . . . pet owners have a top priority in pursuing longer, healthier lives for their pets.”

According to the Veterinary Pet Insurance Company, policyholders spent more than $68 million in 2014 to treat the ten most common medical conditions affecting their pets. For cats, those conditions were bladder or urinary tract disease, periodontitis/dental disease, chronic kidney disease, vomiting/upset stomach, excessive thyroid hormone, diarrhea/intestinal upset, diabetes, inflammatory bowel disease, upper respiratory infection, and lymphoma. For dogs, those conditions were skin allergies, ear infections, non-cancerous skin masses, skin infections, arthritis, vomiting/upset stomach, periodontitis/dental disease, diarrhea/intestinal upset, bladder or urinary tract infection, and soft tissue trauma.

Veterinarians frequently turn to antibiotics, antiparasitics, behavior-modifying drugs, chemotherapeutics, hormones, non-steroidal anti-inflammatories (“NSAIDs”), opioids, sedatives, steroids, and the like to treat a wide variety of conditions encountered by their patients. While increased innovation in the veterinary medicine space brought with it many effective treatments, those treatments often cause severe side-effects, such as anaphylaxis, diarrhea, kidney damage, lethargy, liver damage, skin irritation, ulcers, vomiting, and weakness.

Osteoarthritis is a common disease in domestic animals and is estimated to affect twenty percent of all dogs and ninety percent of all cats over one year of age. The condition is associated with chronic pain and lameness. There are numerous strategies on how to treat osteoarthritis in dogs and cats. They all have similar goals, including giving adequate analgesic effect, preserving joint mobility by slowing down disease progression, and, for some, even improving the pathological condition by restoring the joint (McLaughlin, 2000). NSAIDs are currently the mainstay treatment of osteoarthritis. Although NSAIDs are known to have a strong anti-inflammatory effect, the drugs are also associated with several adverse effects which can cause severe damage, for example in the gastrointestinal tract and the kidneys. There is currently no treatment that completely cures affected animals (KuKanich et al., 2012). There is uncertainty about the safety profile of NSAID treatment, particularly in the long term. There is no NASID labeled for long-term use in cats. Currently there is little treatment available for cats pain long-term.

Veterinarians often turn to NSAIDs for patients with osteoarthritis and to manage pain after surgery and the like. FDA-approved NSAIDs for dogs include carprofen (brand names Carprieve, Carprofen, Novocox, Quellin, Rimadyl, and Vetprofen), deracoxib (brand name Deramaxx), firocoxib (brand name Previcox), grapiprant (brand name Galliprant), meloxicam (brand names loxicom, meloxicam, meloxidyl, and metacam), and robenacoxib (brand name onsior, not approved for long-term use). Most NSAIDs for dogs are given orally or by injection. Only two NSAIDs are FDA-approved for cats: meloxicam (brand names loxicom, meloxicam, and metacam), and robenacoxib (brand name onsior, not approved for long-term use). Meloxicam is approved in cats as a one-time-only injection (given under the cat's skin before surgery) to control pain and inflammation after spaying, neutering, and orthopedic surgery. Robenacoxib is approved for cats to control pain and inflammation after spaying, neutering, and orthopedic surgery and is used once daily (given by mouth or as an injection under the cat's skin) for no more than three days. Currently there are no NSAIDs approved for long-term use in cats.

When cells are damaged, the enzyme cyclooxygenase (“COX”) is activated, which stimulates the production of several substances, including prostaglandins. Prostaglandins contribute to pain, inflammation, and fever. Many NSAIDs work by blocking COX so that fewer prostaglandins are produced. Other NSAIDs work by blocking some activity of certain prostaglandins. While prostaglandins contribute to pain, inflammation, and fever, they also have beneficial effects, including protecting the lining of the stomach and intestines, helping to maintain blood flow to the kidneys, and supporting platelet function (which helps with blood clotting). Because NSAIDs also interfere with the positive functions of prostaglandins, they can cause many negative side effects, some of which are quite severe. The number of adverse drug effects associated with NSAIDs is higher than for any other drug used for pets (The Federal Drug Administration Centre for Veterinary Medicine, 2008). These side effects include death, decreased activity, decreased or no appetite, diarrhea, kidney failure, liver failure, stomach and intestinal perforations, stomach and intestinal ulcers, vomiting. The most common being the gastrointestinal tract, the kidneys, the liver and cardiovascular system (KuKanich et al., 2012). Cats are more sensitive than dogs to the side effects of NSAIDs because they are not able to break down the drug as well as dogs. More than one dose of meloxicam in cats can cause kidney failure or death and the effects of long-term use of other NSAIDs in cats is unknown.

While acetaminophen, which is not an NSAID, is an exceptional pain reliever for people, it is fatal to cats because they lack certain enzymes that the liver needs to break down the drug. While both cats and dogs can develop forms of acetaminophen toxicity, cats are more prone to red blood cell damage and dogs are more likely to get liver damage.

Veterinarians turn to corticosteroids, such as cortisone and synthetic cortisone-like drugs (including dexamethasone, methylprednisolone, prednisone, prednisolone, and triamcinoline) for their potent anti-inflammatory effects. These drugs are often used to reduce allergic, arthritic, and/or dermatologic discomfort. However, such drugs come with serious side effects. Short-term side effects include general loss of energy, increased hunger, increased thirst, increased urination, nausea, panting, vomiting, and worsening infections (especially bacterial skin infections). Long-term side effects include development of adult-onset demodectic mange, development of blackheads, thin hair coat, and thin skin, development of hard plaques or spots on the skin, increased susceptibility to fungal infections (especially in the nasal cavity), increased susceptibility to opportunistic or secondary bacterial infections, muscle weakness, obesity, predisposition to diabetes mellitus, poor wound healing, and urinary tract infections.

Most clinically-relevant opioids have their primary activity at the initial “morphine receptor” or “mu receptors” and are therefore considered “mu agonists.” Veterinary opioids are generally classified as pure mu agonists, partial mu agonists and agonist-antagonists, and mu antagonists. The pure mu agonists are the “strong” opioids, such as fentanyl, hydromorphone, methadone, morphine, and oxymorphone. The partial mu agonists and agonist-antagonists are the “weak” opioids, such as buprenorphine and butorphanol. The mu antagonists are reversal agents, such as naloxone. In acute settings, pure mu agonists can cause respiratory depression, especially when given intravenously. Any opioid can result in dysphoria, which can be difficult to distinguish from the underlying pain sought to be treated. Other negative side effects of opioids include abnormal pain sensitivity, constipation, and gastrointestinal motility problems.

Nutraceuticals, such as glucosamine and chondroitin sulphate, are common supplements given to dogs. These supplements are said to slow down cartilage destruction in the joints while stimulating the synthesis of cartilage in the extracellular matrix (“ECM”) and are also said to have an analgesic effect. Their impact, however, is controversial in that various studies show somewhat different results. Even then, NSAIDs reach higher analgesic effect in less time. See E. Norlund, Pain management in dogs with osteoarthritis, SWEDISH UNIVERSITY OF AGRICULTURAL SCIENCES (2017).

Accordingly, there remains a need for effectively alleviating ailments and/or symptoms of ailments in non-human subjects while concurrently preventing unwanted side-effects of past therapies.

### BRIEF DESCRIPTION OF THE SEVERAL VIEWS OF THE DRAWINGS

FIG. 1 shows both efficacy and side effect ratings when the non-human subject receives a composition comprising: (1) no active agent; (2) HA alone; (3) HMW HA alone; (4) CBD alone; (5) CBD and HA; (6) CBD and HMW HA; (7) Rimadyl; or (8) Meloxicam, as set forth in Example 1.

FIG. 2 shows the ability of embodiments of the invention versus Anipryl® to treat cognitive decline, as set forth in Example 2.

FIG. 3a shows the ability of embodiments of the invention versus Pepcid® to treat unwanted gastrointestinal-specific inflammation in canines and FIG. 3b shows the ability of embodiments of the invention versus Pepcid® to treat unwanted gastrointestinal-specific inflammation in felines, as set forth in Example 3.

FIG. 4a shows the ability of embodiments of the invention versus Cosequin® to treat joint inflammation in canines, FIG. 4b shows the ability of embodiments of the invention versus Cosequin® to treat joint inflammation in felines, and FIG. 4c shows the ability of embodiments of the invention versus Cosequin® to treat joint inflammation in equines, as set forth in Example 4.

FIG. 5a shows the ability of embodiments of the invention versus Clomicalm® to reduce behavior aberrations and anxiety in canines and FIG. 5b shows the ability of embodiments of the invention versus Clomicalm® to reduce behavior aberrations and anxiety in felines, as set forth in Example 5.

FIG. 6 shows the ability of embodiments of the invention versus phenobarbital to treat seizures, as set forth in Example 6.

FIG. 7 shows the ability of embodiments of the invention versus Enalapril to treat cardiac issues in canines, as set forth in Example 7.

FIG. 8 shows the ability of embodiments of the invention versus prednisone to treat skin conditions, as set forth in Example 8.

FIG. 9 shows both efficacy and side effect ratings when the non-human subject receives a composition comprising: (1) no active agent; (2) HA alone; (3) HMW HA alone; (4) CBD alone; (5) CBD and HA; (6) CBD Complex and HMW HA; (7) Rimadyl; or (8) Meloxicam, as set forth in Example 9.

FIG. 10 shows the ability of embodiments of the invention versus Anipryl® to treat cognitive decline, as set forth in Example 10.

FIG. 11a shows the ability of embodiments of the invention versus Pepcid® to treat unwanted gastrointestinal-specific inflammation in canines and FIG. 11b shows the ability of embodiments of the invention versus Pepcid® to treat unwanted gastrointestinal-specific inflammation in felines, as set forth in Example 11.

FIG. 12a shows the ability of embodiments of the invention versus Cosequin® to treat joint inflammation in canines, FIG. 12b shows the ability of embodiments of the invention versus Cosequin® to treat joint inflammation in felines, and FIG. 12c shows the ability of embodiments of the invention versus Cosequin® to treat joint inflammation in equines, as set forth in Example 12.

FIG. 13a shows the ability of embodiments of the invention versus Clomicalm® to reduce behavior aberrations and anxiety in canines and FIG. 13b shows the ability of embodiments of the invention versus Clomicalm® to reduce behavior aberrations and anxiety in felines, as set forth in Example 13.

FIG. 14 shows the ability of embodiments of the invention versus phenobarbital to treat seizures, as set forth in Example 14.

FIG. 15 shows the ability of embodiments of the invention versus Enalapril to treat cardiac issues in canines, as set forth in Example 15.

FIG. 16 shows the ability of embodiments of the invention versus prednisone to treat skin conditions, as set forth in Example 16.

### DETAILED DESCRIPTION OF THE INVENTION

Unless otherwise indicated, terms used herein have the meanings that are commonly-understood by those of ordinary skill in the art.

As used throughout the entire application, the terms “a” and “an” are used in the sense that they mean “at least one,” “at least a first,” “one or more,” or “a plurality” of the referenced components or steps, unless the context clearly dictates otherwise.

The term “about” or “approximately” as used herein means within 20%, preferably within 10%, and more preferably within 5% of a given value or range.

The term “active agent” refers to a compound, composition, molecule, and/or substance effective in the treatment of an ailment.

The term “administering” or any form thereof, such as “administration” and “administered,” refers to the delivery to a subject of a therapeutic agent.

The term “ailment” refers to a disease or disorder, which may be mental and/or physical.

The term “and/or” wherever used herein includes the meaning of “and,” “or,” and “all or any other combination of the elements connected by said term.”

The term “clinical sign” as used herein refers to an observable or measureable condition or behavior in the non-human subject that is indicative of the ailment, condition, disease, and/or symptom. Clinical signs may be those symptoms, conditions, or behaviors that are measured in known or established diagnostic assessments. Non-limiting examples of some clinical signs for atopic dermatitis and allergic dermatitis, that may be used sometimes in such assessments or scoring systems, include: erythema; erosions, excoriations and/or self-induced alopecia; itching, ranging from extremely severe (as demonstrated, in the case of a companion animal such as a dog, by scratching, chewing, licking almost continuously, regardless of what else is happening), to severe (as demonstrated by prolonged episodes of itching while awake, and itching at night and/or while eating, playing or exercising), to moderate (as demonstrated by frequent episodes of itching), to very mild (occasional episodes of itching); hyperpigmentation; lichenification; presence of pustules or epidermal collarets; presence of skin lesions; presence of papules and/or crusts; and/or pruritus.

The term “combination” as used herein refers to any arrangement possible of various components. Such arrangements include mixtures of components as well as separate combinations for concomitant or sequential administration.

As used herein, when used to define products, compositions, and methods, the term “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are open-ended and do not exclude additional, unrecited elements or method steps.

The term “effective amount” means an amount necessary to achieve the desired result.

The term “non-human subject” generally refers to any non-human organism for whom any composition and/or method of the invention is needed or may be beneficial. Typically, the non-human subject is avine, canine, cercopithecine, cricetine, elephantine, equine, feline, glirine, lapine, leporine, macropine, murine, musteline, a non-human primate, otarine, ovine, porcine, psittacine, rodentine, or simian. Examples of non-human subjects include birds, cats, dogs, elephants, ferrets, guinea pigs, hamsters, hedgehogs, horses, kangaroos, llamas, monkeys, mice, otters, pigs, rabbits, rats, seals, and weasels.

The term “one or more” refers to either one or a number above one, e.g., 2, 3, 4, 5, etc. As used herein, a “symptom” of an ailment, disease, and/or condition is any of those symptoms known by a person of ordinary skill in the art as being associated with the ailment, disease, and/or condition. In many cases, a “symptom” of an ailment, disease, and/or condition is also a “clinical sign.”

A “therapeutically-effective amount” corresponds to the amount of composition that is sufficient for producing one or more beneficial results. The therapeutically-effective amount may vary as a function of various parameters, including the ability of the subject to respond to treatment, the age of the subject, the ailment and/or symptom of ailment to be alleviated, the existence and/or nature of concurrent treatment, the frequency of treatment, the mode of administration, and the weight of the subject.

The term “veterinarily-acceptable adjuvant, carrier, and/or vehicle” is intended to include any and all absorption agents, adjuvants (such as alum, lipopolysaccharides, mineral oil emulsions, and saponins), antibacterial agents, antifungal agents, carriers, coatings, diluents, dispersion media, excipients, solvents, and the like that are compatible with administration in non-human subjects.

The compositions of the invention may also contain other veterinarily-acceptable excipients for providing the desired pharmaceutical and/or pharmacodynamics properties, including, for example, clarity, color, isotonicity, modifying and/or maintaining release and/or absorption into the non-human subject, osmolarity, penetration in a particular organ, promoting transport, rate of dissolution, solubility, stability, sterility, and/or viscosity.

Suitable solvent systems may include solvents and oils that are not deleterious to the subject receiving the composition. Suitable solvents may include, but are not limited to, Cremophor EL, dimethylacetamide, dimethyl sulfoxide (“DMSO”), ethanol, glycerin, N-methyl-2-pyrrolidone, polyethylene glycol 300, polyethylene glycol 400, propylene glycol, sorbitol, and mixtures thereof. Suitable examples of oils include arachis, castor, corn, cottonseed, ethyl oleate, olive, glycofurol, isopropyl myristate, palm, peanut, petrolatum, pollock, salmon, sesame, soybean, and combinations thereof.

Solubilization agents include surfactants and complexation agents. Non-limiting examples of surfactants include lecithin, polyoxyethylene sorbitan monolaurate (Tween 20), polyoxyethylene sorbitan monooleate (Tween 80), sorbitan monooleate, polyoxyethylene-polyoxypropylene copolymers, and combinations thereof. Suitable examples of complexation agents include, but are not limited to, arginine, histidine, hydroxypropyl-β-cyclodextrin, lysine, polyvinylpyrrolidone, sulfobutylether-β-cyclodextrin, and combinations thereof.

Stabilization agents include antioxidants, buffers, chelating agents, and combinations thereof. Suitable buffers include, for example, acetate, citrate, phosphate, tartrate, triethanolamine (“TRIS”), and combinations thereof. One or more buffers may be added to adjust the pH of the composition as is appropriate, such as to a range of about 3 to about 5. Suitable antioxidants include, for example, acetylcysteine, ascorbic acid, monothioglycerol, sulfurous acid salts (such as bisulfite and metabisulfite), and combinations thereof. Suitable chelating agents include ethylenediaminetetraacetic acid (“EDTA”), sodium citrate, and combinations thereof.

Tonicity-adjusting agents may be used in the compositions to reduce irritation to the body tissue at the injection site. Suitable tonicity-adjusting agents include, for example, dextrose, glycerin, mannitol, sodium chloride, and combinations thereof.

Antimicrobial agents at any suitable amount may be used in the compositions to prevent microbial growth. Suitable antimicrobial agents include, for example, benzalkonium chloride, benzyl alcohol, chlorobutanol, chlorocresol, meta-cresol, parabens, phenol, phenylmercuric acids (such as acetate, borate, and nitrate), thimerosal, and combinations thereof.

The compositions may be formulated for “non-extended” or “immediate” release. Non-extended release refers to formulations that do not rely on other excipients to delay the release of the active agent(s) from the composition or formulation. The non-extended release formulations do not include components of sustained release formulations such as fat emulsions, microparticles, microspheres, polymer matrix systems, oil-in-water emulsions, and the like.

As used herein, the term “extended-release,” also known as continuous-release (“CTR”), controlled-release (“CR”), modified release (“MR”), sustained-action (“SA”), sustained-release (“SR”), and/or time-release (“TR”), refers to a mechanism used in tablets or capsules to dissolve slowly and release the active agent(s) over time. Advantages of extended-release tablets or capsules include that they can often be taken less frequently than immediate-release formulations and that they keep steadier levels of the drug in the bloodstream, thus extending the duration of the drug action and lowering the peak amount of drug in the bloodstream. In some embodiments, the term “extended-release” refers to a release profile wherein the active agent(s) is released over a period of about 2, 3, 4, 5, 6, 7, 8, 9, 10, 12, 14, 16, 18, 20, 22, or 24 hours, either continuously or in pulses after administration into a non-human subject.

As used herein, the term “delayed-release” refers to a release profile wherein the release of the active agent(s) of composition is delayed or postponed for a given period of time, e.g., about 1, 2, 3, 4, or 5 hours, after administration of the composition.

As used herein, the term “delayed-extended-release” refers to a release profile wherein the release of the active agent(s) of a composition is delayed or postponed for a given period of time, e.g., the lag period of about 1, 2, 3, 4, or 5 hours, after administration of the composition. Once the release starts, the active agent(s) is/are released slowly over time, e.g., over a period of about 2, 3, 4, 5, 6, 7, 8, 9, 10, 12, 14, 16, 18, 20, 22, or 24 hours, either continuously or in pulses.

Sodium hyaluronate is the sodium salt of hyaluronic acid. Sodium hyaluronate is a glycosaminoglycan and long-chain polymer of disaccharide units of Na-glucuronate-N-acetylglucosamine. Sodium hyaluronate is found throughout the extracellular matrix of mammalian connective, epithelial, and neural tissues and in the corneal endothelium. The polyanionic form of sodium hyaluronate is commonly referred to as hyaluronan or hyaluronic acid. As used herein, “hyaluronic acid” or “HA” refers to the polyanionic form of sodium hyaluronate of the following structure:

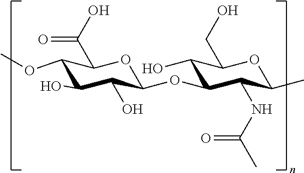

HA is often used as an intra-articular injection to treat knee and ankle pain in patients with osteoarthritis. It is injected into the joint capsule and acts as both a shock absorber and lubricant. It is typically administered as a series of injections, which increase the viscosity of the synovial fluid, which helps lubricate, cushion, and reduce pain in the joint. It may also promote the production of synovial fluid. HA is also used as an aid to ophthalmic surgeries, to coat the bladder lining in treating interstitial cystitis, as dermal fillers to reduce wrinkles on the face and to fill lips, and to assist absorption of biomacromolecules and serve as a nanocarrier when topically applied.

CBD is a phytocannabinoid and among the cannabinoids found in hemp plants. CBD, unlike tetrahydrocannabinol (“THC”), does not cause intoxicating effects. While its mechanism of action is not yet fully known, CBD is believed to interact with a number of biological targets, including cannabinoid receptors and other neurotransmitter receptors. Pharmacological effects of CBD are believed to be mediated through G protein coupled receptors, cannabinoid type I (“CB₁”) and cannabinoid type II (“CB₂”), which are highly expressed in the hippocampus and other parts of the central nervous system (“CNS”). When activated, CB₁ receptors inhibit synaptic transmission through action on voltage-gated calcium and potassium channels, which are known to modulate, e.g., epileptiform and seizure activity. CB₂ receptors are primarily expressed in the immune system, skin, and gastrointestinal lining and have limited expression in the CNS. The effects of CBD are believed to be CB₂ receptor independent. CBD is metabolized by the liver by a number of cytochrome P450 isoenzymes, including CYP2C9, CYP2C19, CYP2D6, and CYP3A4. CBD can be stored for weeks in fatty tissues from which they are slowly released at sub-therapeutic levels back into the blood stream and metabolized via the renal and biliary systems. The main primary metabolite of CBD is 7-hydroxy-cannabidiol.

In addition to activity on CB₁ and CB₂ receptors, there is evidence that CBD also activates 5-HT1A/2A/3A serotonergic and TRPV1-2 vanilloid receptors, antagonizes alpha-1 adrenergic and μ-opioid receptors, inhibits synaptosomal uptake of noradrenaline, dopamine, serotonin, and gamma-aminobutyric acid (“GABA”), and cellular update of anandamide, acts on mitochondria Ca²⁺ stores, blocks low-voltage-activated (T-type) Ca²⁺ channels, stimulates activity of the inhibitory glycine receptor, and inhibits activity of fatty amide hydrolase (“FAAH”). See C. Ibeas Bih et al., Molecular Targets of Cannabidiol in Neurological Disorders, 12(4) NEUROTHERAPEUTICS 699-730 (October 2015); S. Zhornitsky et al., Cannabidiol in humans—the quest for therapeutic targets, 5(5) PHARMACEUTICALS 529-552 (May 21, 2012).

CBD has been viewed as effective in treating certain childhood epilepsy syndromes, such as Dravet Syndrome and Lennox-Gastaut Syndrome, which typically do not respond to conventional anti-seizure medications. CBD was shown to reduce the number of seizures and in some cases stop the seizures completely. The United States Food and Drug Administration recently approved Epidiolex, which contains CBD, for the treatment of Dravet Syndrome and Lennox-Gastaut Syndrome.

CBD is generally well-tolerated, but may cause changes in appetite, diarrhea, irritability, nausea, and tiredness. At room temperature, CBD is a colorless, crystalline solid. CBD is insoluble in water but soluble in organic solvents, such as pentane. Its molecular formula is C₂₁H₃₀O₂ with a molecular weight of 314.469 g/mol. The IUPAC name for CBD is 2-[(1R,6R)-3-methyl-6-prop-1-en-2-ylcyclohex-2-en-1-yl]-5-pentylbenzene-1,3-diol.

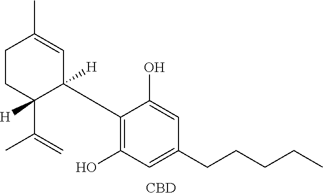

CBD is not scheduled under the Convention on Psychotropic Substances or any other United Nations drug treaty. In 2018, the World Health Organization recommended that CBD remain unscheduled.

Another component of the hemp plant are flavonoids, sometimes referred to as cannaflavins. Flavonoids have been shown to have anti-allergy, anti-bacterial, anti-cancer, anti-inflammatory, and antioxidant effects. Flavonoids include anthocyanin, anthoxanthin, apigenin, cannaflavin A, cannaflavin B, cannaflavin C, catechin, kaempferol, orientin, quercetin, and silymarin.

Yet another component of the hemp plant are terpenes. Terpenes have been shown to inhibit serotonin uptake, enhance norepinepherin activity, increase dopamine, and augment gamma-aminobutyric acid (“GABA”). Terpenes may assist CBD in crossing the blood-brain barrier. Terpenes include aromadendrene, bergamotene, bisabolene, bisabolol, borneol, camphene, carene, caryophyllene, cymene, D3-carene, elemene, eucalyptol, farnesene, fenchol, geraniol, humulene, isopulegol, levomenol, limonene, linalool, myrcene, nerolidol, ocimene, phellandrene, phytol, pinene, pulegone, terpinene, terpineol, terpinolene, and valencene. Caryophyllene has been shown to have anti-inflammatory properties, can act as an analgesic, and can protect cells lining the digestive tract. Limonene has anti-anxiety and anti-depressant activity and has been shown to treat acid reflux. Linalool has been shown to have anti-anxiety and anti-convulsant properties, as well as function as an anesthetic and analgesic. Myrcene can be used as sleep aid and muscle relaxant. Pinene has been shown to have anti-bacterial and anti-inflammatory properties, as well as aid memory and function as a bronchodilator.

“CBD Complex,” as used herein, refers to any of: (1) a combination of CBD and at least one flavonoid; (2) a combination of CBD and at least one terpene; and (3) a combination of CBD and at least one flavonoid and at least one terpene.

Disclosed herein is the inventors' unexpected and surprising invention based upon the synergistic combination of HWM HA and CBD and of HWM HA and CBD Complex, each of which can be formulated into compositions and employed in methods for alleviating at least one ailment and/or symptoms thereof in a non-human subject in need thereof.

### HMW HA and CBD Embodiments

In an embodiment of the invention, the composition comprises HWM HA and CBD. In another embodiment, the composition comprises about 0.2% to about 10.0% HMW HA by weight of the total composition and about 0.3% to about 45% CBD by weight of the total composition. In certain embodiments, the HMW HA is present at about 0.5% to about 5.0% of the total weight of the composition and the CBD is present at about 0.5% to about 25.0% of the total weight of the composition. In other embodiments, the HMW HA is present at about 0.5% to about 3.00% of the total weight of the composition and the CBD is present at about 0.5% to about 15.0% of the total weight of the composition. In a preferred embodiment, the HMW HA is present at about 1.0% of the total weight of the composition and the CBD is present at about 8.0% of the total weight of the composition. In other embodiments, the HMW HA is present at about: 0.2%, 0.3% 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.90%, 8.0%, 8.1%, 8.2%, 8.3%, 8.4%, 8.5%, 8.6%, 8.7%, 8.8%, 8.9%, 9.0/c, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, or 12.0% by weight of the total composition. In other embodiments, the CBD is present at about: 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0% by weight of the total composition.

In an embodiment of the invention, the HMW HA has a molecular weight of at least 900,000 Daltons. In other embodiments of the invention, the HMW HA has a molecular weight of least 1,000,000 Daltons. In another embodiment of the invention, the HMW HA has a molecular weight of least 1,100,000 Daltons.

In embodiments of the invention, the amount of CBD delivered to the non-human subject is about 0.05 mg/kg to about 20 mg/kg. In other embodiments of the invention, the amount of CBD delivered to the non-human subject is about: 0.05 mg/kg, 0.10 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.00 mg/kg, 1.05 mg/kg, 1.10 mg/kg, 1.15 mg/kg, 1.20 mg/kg, 1.25 mg/kg, 1.30 mg/kg, 1.35 mg/kg, 1.40 mg/kg, 1.45 mg/kg, 1.50 mg/kg, 1.55 mg/kg, 1.60 mg/kg, 1.65 mg/kg, 1.70 mg/kg, 1.75 mg/kg, 1.80 mg/kg, 1.85 mg/kg, 1.90 mg/kg, 1.95 mg/kg, 2.00 mg/kg, 2.05 mg/kg, 2.10 mg/kg, 2.15 mg/kg, 2.20 mg/kg, 2.25 mg/kg, 2.30 mg/kg, 2.35 mg/kg, 2.40 mg/kg, 2.45 mg/kg, 2.50 mg/kg, 2.55 mg/kg, 2.60 mg/kg, 2.65 mg/kg, 2.70 mg/kg, 2.75 mg/kg, 2.80 mg/kg, 2.85 mg/kg, 2.90 mg/kg, 2.95 mg/kg, 3.00 mg/kg, 3.05 mg/kg, 3.10 mg/kg, 3.15 mg/kg, 3.20 mg/kg, 3.25 mg/kg, 3.30 mg/kg, 3.35 mg/kg, 3.40 mg/kg, 3.45 mg/kg, 3.50 mg/kg, 3.55 mg/kg, 3.60 mg/kg, 3.65 mg/kg, 3.70 mg/kg, 3.75 mg/kg, 3.80 mg/kg, 3.85 mg/kg, 3.90 mg/kg, 3.95 mg/kg, 4.00 mg/kg, 4.05 mg/kg, 4.10 mg/kg, 4.15 mg/kg, 4.20 mg/kg, 4.25 mg/kg, 4.30 mg/kg, 4.35 mg/kg, 4.40 mg/kg, 4.45 mg/kg, 4.50 mg/kg, 4.55 mg/kg, 4.60 mg/kg, 4.65 mg/kg, 4.70 mg/kg, 4.75 mg/kg, 4.80 mg/kg, 4.85 mg/kg, 4.90 mg/kg, 4.95 mg/kg, 5.00 mg/kg, 5.05 mg/kg, 5.10 mg/kg, 5.15 mg/kg, 5.20 mg/kg, 5.25 mg/kg, 5.30 mg/kg, 5.35 mg/kg, 5.40 mg/kg, 5.45 mg/kg, 5.50 mg/kg, 5.55 mg/kg, 5.60 mg/kg, 5.65 mg/kg, 5.70 mg/kg, 5.75 mg/kg, 5.80 mg/kg, 5.85 mg/kg, 5.90 mg/kg, 5.95 mg/kg, 6.00 mg/kg, 6.05 mg/kg, 6.10 mg/kg, 6.15 mg/kg, 6.20 mg/kg, 6.25 mg/kg, 6.30 mg/kg, 6.35 mg/kg, 6.40 mg/kg, 6.45 mg/kg, 6.50 mg/kg, 6.55 mg/kg, 6.60 mg/kg, 6.65 mg/kg, 6.70 mg/kg, 6.75 mg/kg, 6.80 mg/kg, 6.85 mg/kg, 6.90 mg/kg, 6.95 mg/kg, 7.00 mg/kg, 7.05 mg/kg, 7.10 mg/kg, 7.15 mg/kg, 7.20 mg/kg, 7.25 mg/kg, 7.30 mg/kg, 7.35 mg/kg, 7.40 mg/kg, 7.45 mg/kg, 7.50 mg/kg, 7.55 mg/kg, 7.60 mg/kg, 7.65 mg/kg, 7.70 mg/kg, 7.75 mg/kg, 7.80 mg/kg, 7.85 mg/kg, 7.90 mg/kg, 7.95 mg/kg, 8.00 mg/kg, 8.05 mg/kg, 8.10 mg/kg, 8.15 mg/kg, 8.20 mg/kg, 8.25 mg/kg, 8.30 mg/kg, 8.35 mg/kg, 8.40 mg/kg, 8.45 mg/kg, 8.50 mg/kg, 8.55 mg/kg, 8.60 mg/kg, 8.65 mg/kg, 8.70 mg/kg, 8.75 mg/kg, 8.80 mg/kg, 8.85 mg/kg, 8.90 mg/kg, 8.95 mg/kg, 9.00 mg/kg, 9.05 mg/kg, 9.10 mg/kg, 9.15 mg/kg, 9.20 mg/kg, 9.25 mg/kg, 9.30 mg/kg, 9.35 mg/kg, 9.40 mg/kg, 9.45 mg/kg, 9.50 mg/kg, 9.55 mg/kg, 9.60 mg/kg, 9.65 mg/kg, 9.70 mg/kg, 9.75 mg/kg, 9.80 mg/kg, 9.85 mg/kg, 9.90 mg/kg, 9.95 mg/kg, 10.00 mg/kg, 10.05 mg/kg, 10.10 mg/kg, 10.15 mg/kg, 10.20 mg/kg, 10.25 mg/kg, 10.30 mg/kg, 10.35 mg/kg, 10.40 mg/kg, 10.45 mg/kg, 10.50 mg/kg, 10.55 mg/kg, 10.60 mg/kg, 10.65 mg/kg, 10.70 mg/kg, 10.75 mg/kg, 10.80 mg/kg, 10.85 mg/kg, 10.90 mg/kg, 10.95 mg/kg, 11.00 mg/kg, 11.05 mg/kg, 11.10 mg/kg, 11.15 mg/kg, 11.20 mg/kg, 11.25 mg/kg, 11.30 mg/kg, 11.35 mg/kg, 11.40 mg/kg, 11.45 mg/kg, 11.50 mg/kg, 11.55 mg/kg, 11.60 mg/kg, 11.65 mg/kg, 11.70 mg/kg, 11.75 mg/kg, 11.80 mg/kg, 11.85 mg/kg, 11.90 mg/kg, 11.95 mg/kg, 12.00 mg/kg, etc. up to about 20 mg/kg. In other embodiments of the invention, the amount of CBD delivered to the non-human subject is about 0.25 mg/kg to about 15 mg/kg. In other embodiments of the invention, the amount of CBD delivered to the non-human subject is about 5 mg/kg to about 15 mg/kg. In other embodiments of the invention, the amount of CBD delivered to the non-human subject is about 7 mg/kg to about 10 mg/kg. In another embodiment of the invention, the amount of CBD delivered to the non-human subject is about 8 mg/kg.

In other embodiments of the invention, the composition further comprises at least one additional active agent. In certain embodiments of the invention, the at least one additional active agent is Boswellia, catalase, ginko biloba, glutathione, gotu kola, hawthorn berry, kava, liposomal CoQ10, lutein, nettles, nicotinamide adenine dinucleotide, phosphatidylserine, quercetin, scutillaria, superoxide dismutase (“SOD”), valerian root, or zeaxanthin.

In other embodiments of the invention, the composition further comprises a veterinarily-acceptable preservative.

In yet other embodiments of the invention, the composition further comprises a veterinarily-acceptable flavorant.

Other embodiments of the invention are directed to compositions further comprising a veterinarily-acceptable adjuvant, carrier, and/or vehicle.

In other embodiments of the invention, an effective amount of a composition comprising HMW HA and CBD as detailed above are administered to a non-human subject in need thereof to alleviate at least one ailment and/or symptoms of at least one ailment. In other embodiments, said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian. In other embodiments, said non-human subject is a bird, cat, dog, elephant, ferret, guinea pig, hamster, hedgehog, horse, kangaroo, llama, monkey, mouse, otter, pig, rabbit, rat, seal, or weasel.

In embodiments of the invention, the HMW HA and CBD compositions may be administered by inhalation, intramuscularly, intranasally, intravenously, orally, rectally, subcutaneously, sublingually, transdermally, transmucosally, or by combinations thereof.

In embodiments of the invention, the HMW HA and CBD compositions are provided in aerosol, capsule, cream, dispersion, gel, gelatin capsule, granule, liquid, lotion, lozenge, ointment, paste, patch, pill, powder, solution, spray, suppository, suspension, syrup, and/or tablet (coated or uncoated) form.

In embodiments of the invention, the HMW HA and CBD compositions are administered to alleviate at least one ailment and/or symptoms of such ailments such as arthritis, behavioral disorders, cancers, cardiovascular disorders, cognitive decline, dermatitis, gastrointestinal disorders, inflammation, irritability, nausea, neurological disorders, osteoarthritis, pain, poor appetite, pruritis, seizures, and/or skin disorders. In certain embodiments, said behavioral disorder is aggression, general anxiety, noise phobia, separation anxiety, and/or sleep disturbance. In certain embodiments, said osteoarthritis manifests as ataxia, gait deficit, pain, stiffness, and/or weakness. In certain embodiments, said neurologic disorder manifests as ataxia, disorientation, lameness, pain, paralysis, paresis, seizure, and/or tremors. In certain embodiments, said pain manifests as aggression, decreased appetite, gait deficits, inappropriate eliminations, increased sleeping, lethargy, night restlessness, panting, self segregation, sleep disturbance, and/or vocalizations. In certain embodiments, said cognitive decline manifests as apathy, confusion, house soiling, impaired ability for commands, impaired ability for tasks, impaired ability for work, increased anxiety, increased irritability, negative changes in activity, negative changes in learning, negative changes in memory, negative changes in relationships, negative changes in sleep-wake cycles, negative changes in social behavior, problems with awareness, problems with spatial orientation, and/or repetitive activity. In certain embodiments, said gastrointestinal disorder manifests as anorexia, bloating, constipation, belching, diarrhea, gas, gastroesophageal reflex disease (“GERD”) and symptoms thereof, hiccups, and/or vomiting.

In another embodiment, the HMW HA and CBD compositions are administered such that about 1.0 mg/kg to about 10.0 mg/kg of CBD by weight of said subject is administered. In other embodiments, the HMW HA and CBD compositions are administered such that about 5.0 mg/kg to about 10.0 mg/kg of CBD by weight of said subject is administered. In another embodiment, the HMW HA and CBD compositions are administered such that about 8.0 mg/kg of CBD by weight of said subject is administered.

In certain embodiments, the HMW HA and CBD compositions are administered twice a day for one week and then once a day thereafter. In other embodiments, the HMW HA and CBD compositions are administered at least once a day.

In another embodiment, the HMW HA and CBD compositions are administered orally.

In another embodiment of the invention, provided is a method for treating cognitive decline in a non-human subject, said method comprising administering to said non-human subject in need thereof an effective amount of a composition comprising HMW HA and CBD, wherein said at least one additional active agent is glutathione, ginko biloba, gotu kola, nicotinamide adenine dinucleotide, and/or phosphatidylserine.

In another embodiment of the invention, provided is a method for treating unwanted inflammation in a non-human subject, said method comprising administering to said non-human subject in need thereof an effective amount of a composition comprising HMW HA and CBD, wherein said at least one additional active agent is glutathione, ginko biloba, and/or gotu kola.

In another embodiment of the invention, provided is a method for supporting oxidative stress and/or cell metabolism in a non-human subject, said method comprising administering to said non-human subject in need thereof an effective amount of a composition comprising HMW HA and CBD, wherein said at least one additional active agent is catalase and/or CoQ10.

In another embodiment of the invention, provided is a method for providing joint support to and/or treating unwanted inflammation related to osteoarthritis in a non-human subject, said method comprising administering to said non-human subject in need thereof an effective amount of a composition comprising HMW HA and CBD, wherein said at least one additional active agent is Boswellia and/or superoxide dismutase.

In another embodiment of the invention, provided is a method for treating anxiety and/or behavior disorder and/or sleep disturbance in a non-human subject, said method comprising administering to said non-human subject in need thereof an effective amount of a composition comprising HMW HA and CBD, wherein said at least one additional active agent is kava, scutillaria, and/or valerian root.

In yet another embodiment of the invention, provided is a method for treating seizures in a non-human subject, said method comprising administering to said non-human subject in need thereof an effective amount of a composition comprising HMW HA and CBD, wherein said at least one additional active agent is valerian root and/or scutillaria.

In yet another embodiment of the invention, provided is a method for providing cardiovascular support to a non-human subject, said method comprising administering to said non-human subject in need thereof an effective amount of a composition comprising HMW HA and CBD, wherein said at least one additional active agent is liposomal CoQ10, hawthorn berry, and/or ginko biloba.

In yet another embodiment of the invention, provided is a method for treating at least one acute and/or chronic pruritic skin condition and/or at least one inflammatory skin condition of a non-human subject, said method comprising administering to said non-human subject in need thereof an effective amount of a composition comprising HMW HA and CBD, wherein said at least one additional active agent is quercetin and/or nettles.

In yet another embodiment of the invention, provided is a method for treating declining vision in a non-human subject, said method comprising administering to said non-human subject in need thereof an effective amount of a composition comprising HMW HA and CBD, wherein said at least one additional active agent is lutein and/or zeaxanthin.

HMW HA and CBD Complex Embodiments In an embodiment of the invention, the composition comprises HWM HA and CBD Complex. In another embodiment, the composition comprises about 0.2% to about 10.0% HMW HA by weight of the total composition and CBD Complex, wherein CBD is present at about 0.3% to about 45% by weight of the total composition. In certain embodiments, the HMW HA is present at about 0.5% to about 5.0% of the total weight of the composition and the CBD is present at about 0.5% to about 25.0% of the total weight of the composition. In other embodiments, the HMW HA is present at about 0.5% to about 3.0% of the total weight of the composition and the CBD is present at about 0.5% to about 15.00% of the total weight of the composition. In a preferred embodiment, the HMW HA is present at about 1.0% of the total weight of the HMW HA and CBD Complex composition and the CBD is present at about 8.0% of the total weight of the HMW HA and CBD Complex composition. In other embodiments, the HMW HA is present at about: 0.2%, 0.3% 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, or 12.0% by weight of the total composition. In other embodiments, the CBD is present at about: 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.90%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.00/a, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0/6, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0% by weight of the total composition.

In an embodiment of the invention, the HMW HA has a molecular weight of at least 900,000 Daltons. In other embodiments of the invention, the HMW HA has a molecular weight of least 1,000,000 Daltons. In another embodiment of the invention, the HMW HA has a molecular weight of least 1,100,000 Daltons.

In embodiments of the invention, the amount of CBD delivered to the non-human subject is about 0.05 mg/kg to about 20 mg/kg. In other embodiments of the invention, the amount of CBD delivered to the non-human subject is about: 0.05 mg/kg, 0.10 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.00 mg/kg, 1.05 mg/kg, 1.10 mg/kg, 1.15 mg/kg, 1.20 mg/kg, 1.25 mg/kg, 1.30 mg/kg, 1.35 mg/kg, 1.40 mg/kg, 1.45 mg/kg, 1.50 mg/kg, 1.55 mg/kg, 1.60 mg/kg, 1.65 mg/kg, 1.70 mg/kg, 1.75 mg/kg, 1.80 mg/kg, 1.85 mg/kg, 1.90 mg/kg, 1.95 mg/kg, 2.00 mg/kg, 2.05 mg/kg, 2.10 mg/kg, 2.15 mg/kg, 2.20 mg/kg, 2.25 mg/kg, 2.30 mg/kg, 2.35 mg/kg, 2.40 mg/kg, 2.45 mg/kg, 2.50 mg/kg, 2.55 mg/kg, 2.60 mg/kg, 2.65 mg/kg, 2.70 mg/kg, 2.75 mg/kg, 2.80 mg/kg, 2.85 mg/kg, 2.90 mg/kg, 2.95 mg/kg, 3.00 mg/kg, 3.05 mg/kg, 3.10 mg/kg, 3.15 mg/kg, 3.20 mg/kg, 3.25 mg/kg, 3.30 mg/kg, 3.35 mg/kg, 3.40 mg/kg, 3.45 mg/kg, 3.50 mg/kg, 3.55 mg/kg, 3.60 mg/kg, 3.65 mg/kg, 3.70 mg/kg, 3.75 mg/kg, 3.80 mg/kg, 3.85 mg/kg, 3.90 mg/kg, 3.95 mg/kg, 4.00 mg/kg, 4.05 mg/kg, 4.10 mg/kg, 4.15 mg/kg, 4.20 mg/kg, 4.25 mg/kg, 4.30 mg/kg, 4.35 mg/kg, 4.40 mg/kg, 4.45 mg/kg, 4.50 mg/kg, 4.55 mg/kg, 4.60 mg/kg, 4.65 mg/kg, 4.70 mg/kg, 4.75 mg/kg, 4.80 mg/kg, 4.85 mg/kg, 4.90 mg/kg, 4.95 mg/kg, 5.00 mg/kg, 5.05 mg/kg, 5.10 mg/kg, 5.15 mg/kg, 5.20 mg/kg, 5.25 mg/kg, 5.30 mg/kg, 5.35 mg/kg, 5.40 mg/kg, 5.45 mg/kg, 5.50 mg/kg, 5.55 mg/kg, 5.60 mg/kg, 5.65 mg/kg, 5.70 mg/kg, 5.75 mg/kg, 5.80 mg/kg, 5.85 mg/kg, 5.90 mg/kg, 5.95 mg/kg, 6.00 mg/kg, 6.05 mg/kg, 6.10 mg/kg, 6.15 mg/kg, 6.20 mg/kg, 6.25 mg/kg, 6.30 mg/kg, 6.35 mg/kg, 6.40 mg/kg, 6.45 mg/kg, 6.50 mg/kg, 6.55 mg/kg, 6.60 mg/kg, 6.65 mg/kg, 6.70 mg/kg, 6.75 mg/kg, 6.80 mg/kg, 6.85 mg/kg, 6.90 mg/kg, 6.95 mg/kg, 7.00 mg/kg, 7.05 mg/kg, 7.10 mg/kg, 7.15 mg/kg, 7.20 mg/kg, 7.25 mg/kg, 7.30 mg/kg, 7.35 mg/kg, 7.40 mg/kg, 7.45 mg/kg, 7.50 mg/kg, 7.55 mg/kg, 7.60 mg/kg, 7.65 mg/kg, 7.70 mg/kg, 7.75 mg/kg, 7.80 mg/kg, 7.85 mg/kg, 7.90 mg/kg, 7.95 mg/kg, 8.00 mg/kg, 8.05 mg/kg, 8.10 mg/kg, 8.15 mg/kg, 8.20 mg/kg, 8.25 mg/kg, 8.30 mg/kg, 8.35 mg/kg, 8.40 mg/kg, 8.45 mg/kg, 8.50 mg/kg, 8.55 mg/kg, 8.60 mg/kg, 8.65 mg/kg, 8.70 mg/kg, 8.75 mg/kg, 8.80 mg/kg, 8.85 mg/kg, 8.90 mg/kg, 8.95 mg/kg, 9.00 mg/kg, 9.05 mg/kg, 9.10 mg/kg, 9.15 mg/kg, 9.20 mg/kg, 9.25 mg/kg, 9.30 mg/kg, 9.35 mg/kg, 9.40 mg/kg, 9.45 mg/kg, 9.50 mg/kg, 9.55 mg/kg, 9.60 mg/kg, 9.65 mg/kg, 9.70 mg/kg, 9.75 mg/kg, 9.80 mg/kg, 9.85 mg/kg, 9.90 mg/kg, 9.95 mg/kg, 10.00 mg/kg, 10.05 mg/kg, 10.10 mg/kg, 10.15 mg/kg, 10.20 mg/kg, 10.25 mg/kg, 10.30 mg/kg, 10.35 mg/kg, 10.40 mg/kg, 10.45 mg/kg, 10.50 mg/kg, 10.55 mg/kg, 10.60 mg/kg, 10.65 mg/kg, 10.70 mg/kg, 10.75 mg/kg, 10.80 mg/kg, 10.85 mg/kg, 10.90 mg/kg, 10.95 mg/kg, 11.00 mg/kg, 11.05 mg/kg, 11.10 mg/kg, 11.15 mg/kg, 11.20 mg/kg, 11.25 mg/kg, 11.30 mg/kg, 11.35 mg/kg, 11.40 mg/kg, 11.45 mg/kg, 11.50 mg/kg, 11.55 mg/kg, 11.60 mg/kg, 11.65 mg/kg, 11.70 mg/kg, 11.75 mg/kg, 11.80 mg/kg, 11.85 mg/kg, 11.90 mg/kg, 11.95 mg/kg, 12.00 mg/kg, etc. up to about 20 mg/kg. In other embodiments of the invention, the amount of CBD delivered to the non-human subject is about 0.25 mg/kg to about 15 mg/kg. In other embodiments of the invention, the amount of CBD delivered to the non-human subject is about 5 mg/kg to about 15 mg/kg. In other embodiments of the invention, the amount of CBD delivered to the non-human subject is about 7 mg/kg to about 10 mg/kg. In another embodiment of the invention, the amount of CBD delivered to the non-human subject is about 8 mg/kg

In other embodiments of the invention, the composition further comprises at least one additional active agent. In certain embodiments of the invention, the at least one additional active agent is Boswellia, catalase, ginko biloba, glutathione, gotu kola, hawthorn berry, kava, liposomal CoQ10, lutein, nettles, nicotinamide adenine dinucleotide, phosphatidylserine, quercetin, scutillaria, superoxide dismutase (“SOD”), valerian root, or zeaxanthin.

In other embodiments of the invention, the composition further comprises a veterinarily-acceptable preservative.

In yet other embodiments of the invention, the composition further comprises a veterinarily-acceptable flavorant.

Other embodiments of the invention are directed to compositions further comprising a veterinarily-acceptable adjuvant, carrier, and/or vehicle.

In other embodiments of the invention, an effective amount of a composition comprising HMW HA and CBD Complex as detailed above are administered to a non-human subject in need thereof to alleviate at least one ailment and/or symptoms of at least one ailment.

In other embodiments, said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian. In other embodiments, said non-human subject is a bird, cat, dog, elephant, ferret, guinea pig, hamster, hedgehog, horse, kangaroo, llama, monkey, mouse, otter, pig, rabbit, rat, seal, or weasel.

In embodiments of the invention, the HMW HA and CBD Complex compositions may be administered by inhalation, intramuscularly, intranasally, intravenously, orally, rectally, subcutaneously, sublingually, transdermally, transmucosally, or by combinations thereof.

In embodiments of the invention, the HMW HA and CBD Complex compositions are provided in aerosol, capsule, cream, dispersion, gel, gelatin capsule, granule, liquid, lotion, lozenge, ointment, paste, patch, pill, powder, solution, spray, suppository, suspension, syrup, and/or tablet (coated or uncoated) form.

In embodiments of the invention, the HMW HA and CBD Complex compositions are administered to alleviate at least one ailment and/or symptoms of such ailments such as arthritis, behavioral disorders, cancers, cardiovascular disorders, cognitive decline, dermatitis, gastrointestinal disorders, inflammation, irritability, nausea, neurological disorders, osteoarthritis, pain, poor appetite, pruritis, seizures, and/or skin disorders. In certain embodiments, said behavioral disorder is aggression, general anxiety, noise phobia, separation anxiety, and/or sleep disturbance. In certain embodiments, said osteoarthritis manifests as ataxia, gait deficit, pain, stiffness, and/or weakness. In certain embodiments, said neurologic disorder manifests as ataxia, disorientation, lameness, pain, paralysis, paresis, seizure, and/or tremors. In certain embodiments, said pain manifests as aggression, decreased appetite, gait deficits, inappropriate eliminations, increased sleeping, lethargy, night restlessness, panting, self segregation, sleep disturbance, and/or vocalizations. In certain embodiments, said cognitive decline manifests as apathy, confusion, house soiling, impaired ability for commands, impaired ability for tasks, impaired ability for work, increased anxiety, increased irritability, negative changes in activity, negative changes in learning, negative changes in memory, negative changes in relationships, negative changes in sleep-wake cycles, negative changes in social behavior, problems with awareness, problems with spatial orientation, and/or repetitive activity. In certain embodiments, said gastrointestinal disorder manifests as anorexia, bloating, constipation, belching, diarrhea, gas, gastroesophageal reflex disease (“GERD”) and symptoms thereof, hiccups, and/or vomiting.

In another embodiment, the HMW HA and CBD Complex compositions are administered such that about 1.0 mg/kg to about 10.0 mg/kg of CBD by weight of said subject is administered. In other embodiments, the HMW HA and CBD Complex compositions are administered such that about 5.0 mg/kg to about 10.0 mg/kg of CBD by weight of said subject is administered. In another embodiment, the HMW HA and CBD Complex compositions are administered such that about 8.0 mg/kg of CBD by weight of said subject is administered.

In certain embodiments, the HMW HA and CBD Complex compositions are administered twice a day for one week and then once a day thereafter. In other embodiments, the HMW HA and CBD Complex compositions are administered at least once a day.

In another embodiment, the HMW HA and CBD Complex compositions are administered orally.

In another embodiment of the invention, provided is a method for treating cognitive decline in a non-human subject, said method comprising administering to said non-human subject in need thereof an effective amount of a composition comprising HMW HA and CBD Complex, wherein said at least one additional active agent is glutathione, ginko biloba, gotu kola, nicotinamide adenine dinucleotide, and/or phosphatidylserine.

In another embodiment of the invention, provided is a method for treating unwanted inflammation in a non-human subject, said method comprising administering to said non-human subject in need thereof an effective amount of a composition comprising HMW HA and CBD Complex, wherein said at least one additional active agent is glutathione, ginko biloba, and/or gotu kola.

In another embodiment of the invention, provided is a method for supporting oxidative stress and/or cell metabolism in a non-human subject, said method comprising administering to said non-human subject in need thereof an effective amount of a composition comprising HMW HA and CBD Complex, wherein said at least one additional active agent is catalase and/or CoQ10.

In another embodiment of the invention, provided is a method for providing joint support to and/or treating unwanted inflammation related to osteoarthritis in a non-human subject, said method comprising administering to said non-human subject in need thereof an effective amount of a composition comprising HMW HA and CBD Complex, wherein said at least one additional active agent is Boswellia and/or superoxide dismutase.

In another embodiment of the invention, provided is a method for treating anxiety and/or behavior disorder and/or sleep disturbance in a non-human subject, said method comprising administering to said non-human subject in need thereof an effective amount of a composition comprising HMW HA and CBD Complex, wherein said at least one additional active agent is kava, scutillaria, and/or valerian root.

In yet another embodiment of the invention, provided is a method for treating seizures in a non-human subject, said method comprising administering to said non-human subject in need thereof an effective amount of a composition comprising HMW HA and CBD Complex, wherein said at least one additional active agent is valerian root and/or scutillaria.

In yet another embodiment of the invention, provided is a method for providing cardiovascular support to a non-human subject, said method comprising administering to said non-human subject in need thereof an effective amount of a composition comprising HMW HA and CBD Complex, wherein said at least one additional active agent is liposomal CoQ10, hawthorn berry, and/or ginko biloba.

In yet another embodiment of the invention, provided is a method for treating at least one acute and/or chronic pruritic skin condition and/or at least one inflammatory skin condition of a non-human subject, said method comprising administering to said non-human subject in need thereof an effective amount of a composition comprising HMW HA and CBD Complex, wherein said at least one additional active agent is quercetin and/or nettles.

In yet another embodiment of the invention, provided is a method for treating declining vision in a non-human subject, said method comprising administering to said non-human subject in need thereof an effective amount of a composition comprising HMW HA and CBD Complex, wherein said at least one additional active agent is lutein and/or zeaxanthin.

Compositions of the invention may be formulated in any conventional manner using one or more veterinarily-acceptable carriers comprising excipients and auxiliaries, which facilitate processing of the active compound(s) into preparations. Proper formulation is dependent upon the route of administration chosen. Veterinarily-acceptable carriers and excipients are generally known to those skilled in the art and examples are set forth in, e.g., REMINGTON'S THE SCIENCE AND PRACTICE OF PHARMACY.

If administered more than once, the route of administration for the first administration can be different from the route of administration for the second administration. For example, the route of administration for the first administration may be parenteral and the route of administration for the second administration may be oral.

For all amounts listed herein, including in the claims, a listed range means not only the listed numbers but each and every number within that range, including all increments. For example, a range of “about 1 to about 3” means about 1.1, about 1.2, about 1.3, about 1.4, about 1.5, about 1.6, about 1.7, about 1.8, about 1.9, about 2.0. about 2.1, about 2.2, about 2.3, about 2.4, about 2.5, about 2.6, about 2.7, about 2.8, about 2.9, and about 3.0, as well as all smaller increments therein.

### EXAMPLES

Embodiments of the invention are illustrated by the following Examples. The invention is not limited to following embodiments, which are merely exemplary.

### Example 1—Pain Relief and Side Effects

This example examines the ability of various compositions to minimize pain and examines the level of adverse side effects of each composition.

Inclusion Criteria

(1) Canines over one year of age.

(2) Radiographic evidence of osteoarthritis as determined by the managing veterinarian.

(3) No other co-morbidities.

(4) Sufficient wash-out of any other nutraceutical and/or pharmaceutical and no further use of any other nutraceutical and/or pharmaceutical until completion of study.

Exclusion Criteria

Owner cannot comply with evaluation and/or follow-up requirements.

Materials and Methods

At the outset of the study, participants receive a full physical examination, including weight measurement, thigh circumference, and CBC/blood chemistries. Because osteoarthritis is not only a disease of pain, but one characterized by behavioral, appetite, anxiety/aggression, family socialization, and sleep aberrations, the pet's owners are familiarized with the pain scale (canine orthopedic index) and additional behavioral assessment log.

The patient group is twenty-four dogs suffering from osteoarthritis, broken into eight groups of three dogs. The first group receives a control composition with no active agent; the second group receives a composition comprising HA as the sole active agent; the third group receives a composition comprising HMW HA as the sole active agent; the fourth group receives a composition comprising CBD as the sole active agent; the fifth group receives a composition comprising HA and CBD as the active agents; the sixth group receives a composition comprising HMW HA and CBD as the active agents; the seventh group receives a composition comprising Rimadyl as the active agent; and the eighth group receives a composition comprising Meloxicam as the active agent.

Each composition is administered for a period of four (4) weeks. The owner completes the electronic log questionnaire each week. At the end of the 4-week study, participants have another full physical examination, including weight measurement, thigh circumference, and CBC/blood chemistries, with an exit interview of the owners to gain final impressions.

Results

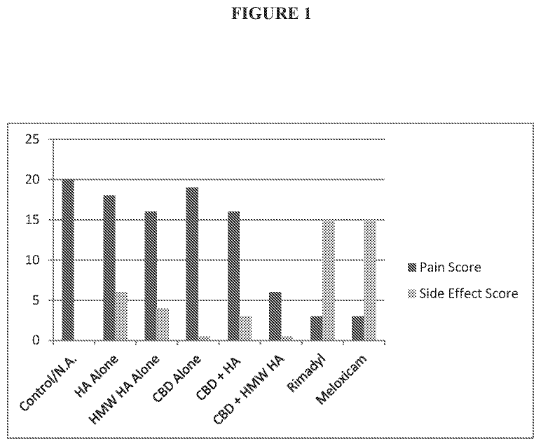

Each group is evaluated for pain scores and the scores averaged. The following scores are observed and the results depicted in FIG. 1:

| Group | Active | Average | Average Side |
|---|---|---|---|
| # | Substance(s) | Pain Score | Effect Score |
| 1 | Control/Not Applicable | 20 | 0 |
| 2 | HA Alone | 18 | 6 |
| 3 | HMW HA Alone | 16 | 4 |
| 4 | CBD Alone | 19 | 0.5 |
| 5 | CBD + HA | 16 | 3 |
| 6 | CBD + HMW HA | 6 | .5 |
| 7 | Rimadyl | 3 | 15 |
| 8 | Meloxicam | 3 | 15 |

As is evident, conventional treatments for osteoarthritis of Rimadyl and Meloxicam are effective in terms of reducing pain, but bring a high degree of adverse side effects. HA alone and HMW HA alone have a low degree of adverse side effects, but only slightly reduce pain. CBD alone and CBD with HA have an even lower degree of adverse side effects than do HA alone and HMW HA alone, but are not especially effective in reducing pain.

However, unexpected and surprising results are observed with the CBD and HMW HA composition, which greatly reduces pain and brings a very low degree of adverse side effects.

### Example 2—Effects of HMW HA and CBD Compositions on Treating Cognitive Decline

Selegiline (marketed as Anipryl®) is currently the only FDA-approved treatment for canine cognitive dysfunction. Although the onset of action can be variable, ranging from four to twelve weeks, most dogs show some improvement after one month of treatment and there may be continued treatment over time.

This example examines the ability of embodiments of the invention versus Anipryl® to treat cognitive decline.

Materials and Methods

The patient group is twenty dogs suffering from cognitive dysfunction syndrome, as determined by the Cognitive Dysfunction Screening Checklist. See G. Landsberg et al., 2003 HANDBOOK OF BEHAVIOR PROBLEMS OF THE DOG AND CAT (Elsevier Science Limited) (2003). The twenty dogs are broken into five groups of four dogs. The first group receives Anipryl® as the sole active agent; the second group receives a composition comprising HMW HA as the sole active agent; the third group receives a composition comprising CBD as the sole active agent; the fourth group receives a composition comprising HMW HA and CBD as the active agents; and the fifth group receives a composition comprising HMW HA, CBD, nicotinamide adenine dinucleotide (“NAD”), and phosphatidylserine as the active agents.

Each composition is administered for a period of sixty (60) days, twice per day (“BID”). The owner completes the Checklist on days 0 (“Initial Score”), 15, 30, 45, and 60 (“Final Score”) of treatment.

Results

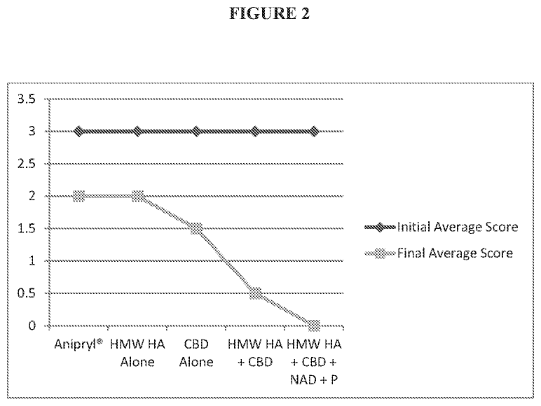

Each group is evaluated and Checklist scores averaged. The following scores are observed and the results depicted in FIG. 2:

| Group | | Average | Average |
|---|---|---|---|
| # | Active Substance(s) | Initial Score | Final Score |
| 1 | Anipryl ® | 3 | 2 |
| 2 | HMW HA Alone | 3 | 2 |
| 3 | CBD Alone | 3 | 1.5 |
| 4 | HMW HA + CBD | 3 | .5 |
| 5 | HMW HA + CBD + NAD + | 3 | 0 |
| | phosphatidylserine | | |

As is evident, Anipryl® is as effective as HMW HA alone in terms of reducing cognitive decline and CBD alone is even more effective. However, unexpected and surprising results are observed with the HMW HA+CBD (which lowers the score from 3 to 0.5) and HMW HA+CBD+NAD+phosphatidylserine composition (which lowers the score from 3 to 0).

### Example 3—Effects of HMW HA and CBD Compositions on Treating Unwanted Gastrointestinal-Specific Inflammation

Famotidine, commonly marketed as Pepcid®, is used to help reduce the amount of stomach acid produced and can be used to treat gastritis, esophagitis, and gastric or esophageal reflux, as well as to prevent stomach or duodenal ulcers in animals with kidney failure.

This example examines the ability of embodiments of the invention versus Pepcid® to treat unwanted GI-specific inflammation characterized by gastritis, reflux, vomiting, diarrhea, and inappetence.

Materials and Methods

The first patient group is twenty dogs suffering from GI-specific inflammation. The second patient group is twenty cats suffering from GI-specific inflammation. Each patient group is broken into five groups of four animals. The first groups receive Pepcid® as the sole active agent; the second groups receive a composition comprising HMW HA as the sole active agent; the third groups receive a composition comprising CBD as the sole active agent; the fourth groups receive a composition comprising HMW HA and CBD as the active agents; and the fifth groups receive a composition comprising HMW HA, CBD, and glutathione as the active agents.

Each composition is administered for a period of twenty-eight (28) days, BID. The owner observes potential reduction of symptoms at the end of treatment and rates the reduction as no reduction (score of 0), mild reduction (score of 1), moderate reduction (score of 2), significant reduction (score of 3), or resolution of symptoms (score of 4).

Results

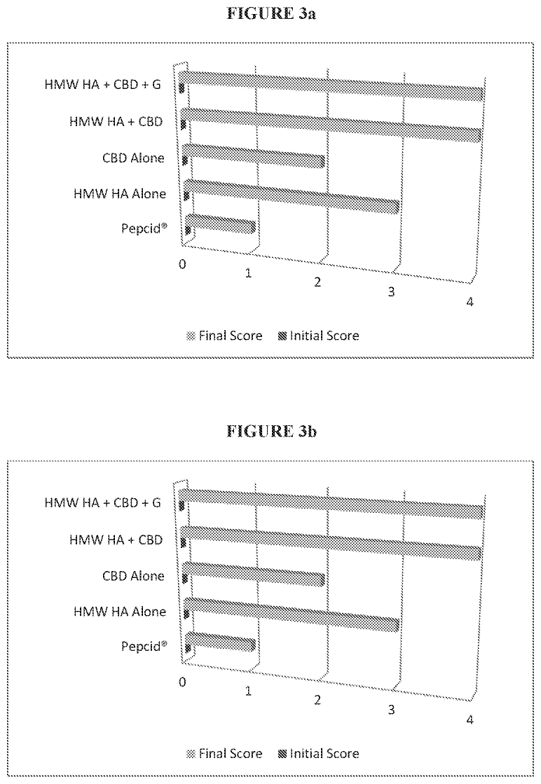

The following canine scores are observed and the results depicted in FIG. 3a:

| Group | | | |
|---|---|---|---|
| # | Active Substance(s) | Initial Score | Final Score |
| 1 | Pepcid ® | 0 | 1 |
| 2 | HMW HA Alone | 0 | 3 |
| 3 | CBD Alone | 0 | 2 |
| 4 | HMW HA + CBD | 0 | 4 |
| 5 | HMW HA + CBD + | 0 | 4 |
| | glutathione | | |

The following feline scores are observed and the results depicted in FIG. 3b:

| Group | | | |
|---|---|---|---|
| # | Active Substance(s) | Initial Score | Final Score |
| 1 | Pepcid ® | 0 | 1 |
| 2 | HMW HA Alone | 0 | 3 |
| 3 | CBD Alone | 0 | 2 |
| 4 | HMW HA + CBD | 0 | 4 |
| 5 | HMW HA + CBD + | 0 | 4 |
| | glutathione | | |

As is evident, compositions with HMW HA and CBD as well as compositions with HMW HA, CBD, and glutathione are unexpectedly and surprisingly more effective than Pepcid® at reducing symptoms of unwanted GI-specific inflammation.

### Example 4—Effects of HMW HA and CBD Compositions on Treating Joint Inflammation

Cosequin®, a supplement containing TRH122®, chondroitin sulfate, FCHG49®, glucosamine, and manganese ascorbate, is a patented, scientifically-researched nutritional supplement dispensed by thousands of veterinarians to help dogs maintain healthy joints.

This example examines the ability of embodiments of the invention versus Cosequin® to treat joint inflammation.

Materials and Methods

The first patient group is twenty dogs suffering from joint inflammation. The second patient group is twenty cats suffering from joint inflammation. The third patient group is twenty horses suffering from joint inflammation. Each patient group is broken into five groups of four animals. The first groups receive Cosequin® as the sole active agent; the second groups receive a composition comprising HMW HA as the sole active agent; the third groups receive a composition comprising CBD as the sole active agent; the fourth groups receive a composition comprising HMW HA and CBD as the active agents; and the fifth groups receive a composition comprising HMW HA, CBD, Boswellia, and superoxide dismutase (“SOD”) as the active agents.

Each composition is administered for a period of sixty (60) days, BID. Efficacy is determined by a decrease in body temperature over the inflamed joint, as measured by thermal imaging (e.g., via Digatherm device). Thermal imaging is performed on days 0 (“Initial Score”), 15, 30, 45, and 60 (“Final Score”) of treatment.

Results

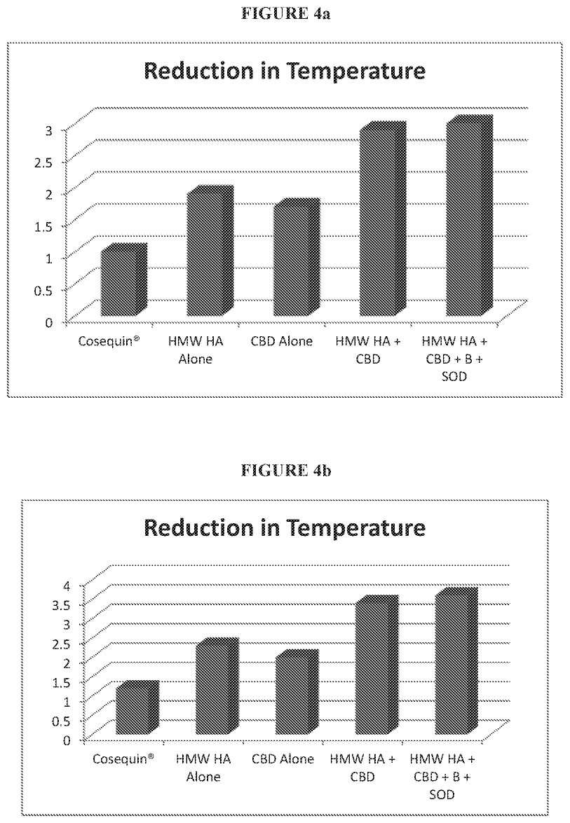

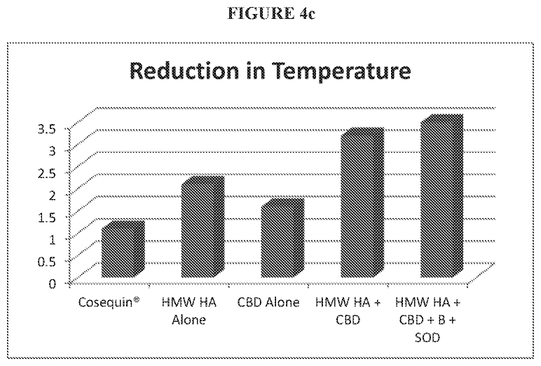

The following canine scores are observed and the results depicted in FIG. 4a:

| Group | | Reduction in |
|---|---|---|
| # | Active Substance(s) | Temperature (° F.) |
| 1 | Cosequin ® | 1 |
| 2 | HMW HA Alone | 1.9 |
| 3 | CBD Alone | 1.7 |
| 4 | HMW HA + CBD | 2.9 |
| 5 | HMW HA + CBD + | 3.0 |
| | Boswellia + SOD | |

The following feline scores are observed and the results depicted in FIG. 4b:

| Group | | Reduction in |
|---|---|---|
| # | Active Substance(s) | Temperature (° F.) |
| 1 | Cosequin ® | 1.2 |
| 2 | HMW HA Alone | 2.3 |
| 3 | CBD Alone | 2.0 |
| 4 | HMW HA + CBD | 3.4 |
| 5 | HMW HA + CBD + | 3.6 |
| | Boswellia + SOD | |

The following equine scores are observed and the results depicted in FIG. 4c:

| Group | | Reduction in |
|---|---|---|
| # | Active Substance(s) | Temperature (° F.) |
| 1 | Cosequin ® | 1.1 |
| 2 | HMW HA Alone | 2.1 |
| 3 | CBD Alone | 1.6 |
| 4 | HMW HA + CBD | 3.2 |
| 5 | HMW HA + CBD + | 3.5 |
| | Boswellia + SOD | |

As is evident, compositions with HMW HA and CBD as well as compositions with HMW HA, CBD, Boswellia, and SOD are unexpectedly and surprisingly more effective than Cosequin® at reducing joint inflammation.

### Example 5—Effects of HMW HA and CBD Compositions on Reducing Behavior Aberrations and Anxiety

Clomicalm® is used in dogs to treat behavior problems such as separation anxiety, excessive barking, and destructive behavior. Clomicalm® is used in cats to help treat behavior problems such as urine spraying, certain types of aggression, and compulsive behaviors (such as excessive grooming).

This example examines the ability of embodiments of the invention versus Clomicalm® to reduce behavior aberrations and anxiety.

Materials and Methods

The first patient group is twenty dogs suffering behavior aberrations and/or anxiety, as determined by the canine behavioral assessment and research questionnaire (“C-BARQ”). See, e.g., P. Wiener et al., Use of questionnaire-based data to assess dog personality, 16 JOURNAL OF VETERINARY BEHAVIOR 81-85 (2016). The second patient group is twenty cats suffering behavior aberrations and/or anxiety, as determined by the feline behavioral assessment and research questionnaire (“Fe-BARQ”).

Each group is broken into five groups of four animals. The first group receives Clomicalm® as the sole active agent; the second group receives a composition comprising HMW HA as the sole active agent; the third group receives a composition comprising CBD as the sole active agent; the fourth group receives a composition comprising HMW HA and CBD as the active agents; and the fifth group receives a composition comprising HMW HA, CBD, valerian root, and scutillaria as the active agents.

Each composition is administered for a period of sixty (60) days, once a day (“SID”). Owners complete the species-appropriate C-BARQ or Fe-BARQ on days 0 and 60 of treatment. The results from the overall questionnaire are averaged and characterized as no improvement (score of 0), mild improvement (score of 1), moderate improvement (score of 2), or marked improvement (score of 3).

Results

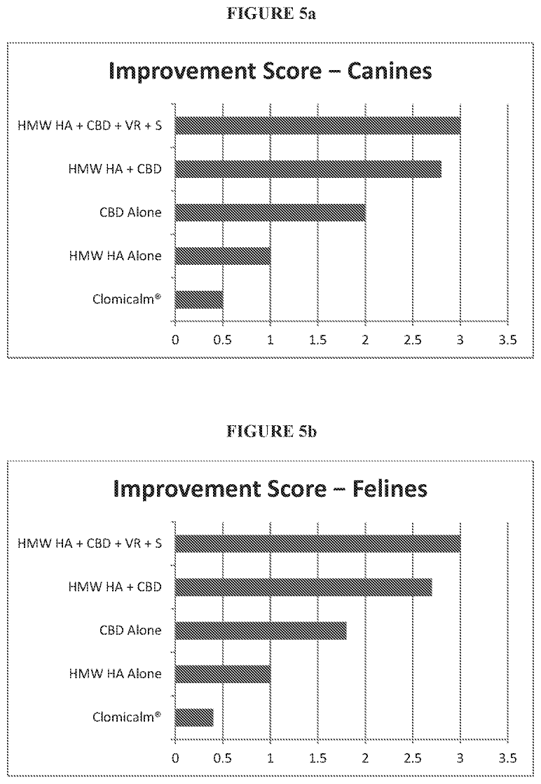

The following canine scores are observed and the results depicted in FIG. 5a:

| Group | | Improvement |
|---|---|---|
| # | Active Substance(s) | Score |
| 1 | Clomicalm ® | .5 |
| 2 | HMW HA Alone | 1 |
| 3 | CBD Alone | 2 |
| 4 | HMW HA + CBD | 2.8 |
| 5 | HMW HA + CBD + | 3 |
| | Valerian Root + Scutillaria | |

The following feline scores are observed and the results depicted in FIG. 5b:

| | | Improvement |
|---|---|---|
| Group # | Active Substance(s) | Score |
| 1 | Clomicalm ® | .4 |
| 2 | HMW HA Alone | 1 |
| 3 | CBD Alone | 1.8 |
| 4 | HMW HA + CBD | 2.7 |
| 5 | HMW HA + CBD + Valerian Root + | 3 |
| | Scutillaria | |

As is evident, compositions with HMW HA and CBD as well as compositions with HMW HA, CBD, valerian root, and scutillaria are unexpectedly and surprisingly more effective than Clomicalm® at reducing behavior aberrations and anxiety.

### Example 6—Effects of HMW HA and CBD Compositions on Treating Seizures

Phenobarbital, while not FDA-approved for use in dogs, is the primary medication used to treat seizures in dogs. Phenobarbital is inexpensive, easy to use, and augments the activity of gamma-aminobutyric acid (“GABA”) in the brain, which stops signals from passing from one neuron to the next. Taking phenobarbital daily stops seizures in dogs by reducing electrical energy. Understandably, many side effects of phenobarbital are neurological, making the dog lethargic, sedated, restless, hyper-excited, and/or uncoordinated. These side effects may or may not resolve after several weeks if the dog's system becomes used to the medication. Long-term side effects of phenobarbital include frequent urination, excessive drinking, and excessive eating (which, in turn, can result in weight gain). The most dangerous side effect of phenobarbital is liver damage. While it occurs in a small percentage of dogs, long-term use of phenobarbital can case scarring of the liver and subsequent liver failure.

This example examines the ability of embodiments of the invention versus phenobarbital to treat seizures.

Materials and Methods

The patient group is twenty-four dogs suffering from seizures as diagnosed by their referring veterinarian. The patient group is broken into six groups of four animals. The first group receives phenobarbital as the sole active agent; the second group receives a composition comprising phenobarbital and Keppra®; the third group receives a composition comprising HMW HA as the sole active agent; the fourth group receives a composition comprising CBD as the sole active agent; the fifth group receives a composition comprising HMW HA and CBD as the active agents; and the sixth group receives a composition comprising HMW HA, CBD, valerian root, and scutillaria as the active agents.

Each composition is administered for a period of sixty (60) days, BID. Each dog is evaluated on days 0 (“Initial Score”), 15, 30, 45, and 60 (“Final Score”) of treatment. The results are averaged and characterized on a scale of 1 (seizure-free) to 10 (daily seizures or multiple seizures during the event).

Results

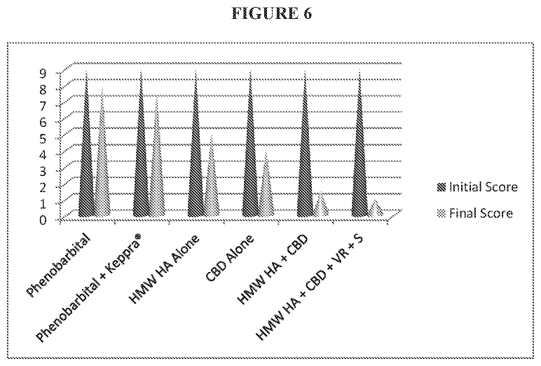

The following canine scores are observed and the results depicted in FIG. 6:

| | | Initial | Final |
|---|---|---|---|
| Group # | Active Substance(s) | Score | Score |
| 1 | Phenobarbital | 9 | 8 |
| 2 | Phenobarbital + Keppra ® | 9 | 7.5 |
| 3 | HMW HA Alone | 9 | 5 |
| 4 | CBD Alone | 9 | 4 |
| 5 | HMW HA + CBD | 9 | 1.5 |
| 6 | HMW HA + CBD + Valerian Root + | 9 | 1 |
| | Scutillaria | | |

As is evident, compositions with HMW HA and CBD as well as compositions with HMW HA, CBD, valerian root, and scutillaria are unexpectedly and surprisingly more effective than phenobarbital and phenobarbital and Keppra® at reducing seizures.

### Example 7—Effects of HMW HA and CBD Compositions on Cardiac Function

Enalapril is an angiotensin-converting enzyme (“ACE”) inhibitor that is commonly prescribed by veterinarians for the treatment of cardiac issues in dogs. Enalapril relaxes blood vessels, which allows blood to flow more freely, reduce blood pressure, and reduce the heart's workload. Enalapril is useful for treating heart failure, heart murmurs, and high blood pressure. Side effects with enalapril include allergic reaction (resulting in hives and/or labored breathing), vomiting, diarrhea, decreased appetite, dizziness, fainting, digestive tract ulcers, high potassium levels, fever, and lethargy.

This example examines the ability of embodiments of the invention versus enalapril to treat heart failure.

Materials and Methods

The patient group is twenty-four dogs suffering from heart failure.

The patient group is broken into six groups of four dogs. The first group receives enalapril as the sole active agent; the second group receives a composition comprising enalapril and Lasix®; the third group receives a composition comprising HMW HA as the sole active agent; the fourth group receives a composition comprising CBD as the sole active agent; the fifth group receives a composition comprising HMW HA and CBD as the active agents; and the sixth group receives a composition comprising HMW HA, CBD, liposomal CoQ10, hawthorn berry, and ginko biloba as the active agents.

Each composition is administered for a period of ninety (90) days, BID. Each animal is evaluated on days 0 (“Initial Score”), 30, 60, and 90 (“Final Score”) of treatment. The results are averaged and characterized on a scale of 1 (few to no clinical signs of heart failure) to 10 (severe heart failure).

Results

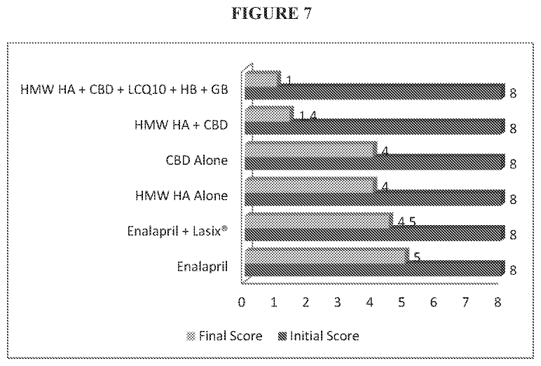

The following canine scores are observed and the results depicted in FIG. 7:

| | | Initial | Final |
|---|---|---|---|
| Group # | Active Substance(s) | Score | Score |
| 1 | Enalapril | 8 | 5 |
| 2 | Enalapril + Lasix ® | 8 | 4.5 |
| 3 | HMW HA Alone | 8 | 4 |
| 4 | CBD Alone | 8 | 4 |
| 5 | HMW HA + CBD | 8 | 1.4 |
| 6 | HMW HA + CBD + liposomal | 8 | 1 |
| | CoQ10 + hawthorn berry + | | |
| | ginko biloba | | |

As is evident, compositions with HMW HA and CBD as well as compositions with HMW HA, CBD, liposomal CoQ10, hawthorn berry, and ginko biloba are unexpectedly and surprisingly more effective than enalapril and enalapril and Lasix® at treating heart failure.

### Example 8—Effects of HMW HA and CBD Compositions on Skin Conditions

Prednisone is a corticosteroid commonly used to treat dogs who suffer from skin allergies and autoimmune-based skin diseases. Side effects of prednisone include increased thirst and hunger, panting, loss of energy, vomiting, and/or skin infections. Long-term side effects of prednisone use include obesity (due to increased hunger), inability to heal fully from infection, urinary tract infections, development of hard calcium deposits on the skin, muscle weakness, distended stomach, and diabetes. Liver damage and iatrogenic Cushing's disease are the most serious side effects of prednisone use.

This example examines the ability of embodiments of the invention versus prednisone to treat skin conditions.

Materials and Methods

The patient group is twenty-four dogs with a history of severe, chronic skin issues, atopy, pruritis, yeast overgrowth, and/or infection, as diagnosed by their referring veterinarian. The patient group is broken into six groups of four animals. The first group receives prednisone as the sole active agent; the second group receives a composition comprising prednisone and HMW HA as the active agents; the third group receives a composition comprising HMW HA as the sole active agent; the fourth group receives a composition comprising CBD as the sole active agent; the fifth group receives a composition comprising HMW HA and CBD as the active agents; and the sixth group receives a composition comprising HMW HA, CBD, quercetin, and nettles as the active agents.

Each composition is administered for a period of sixty (60) days, BID. Each dog is evaluated on days 0 (“Initial Score”), 20, 40, and 60 (“Final Score”) of treatment. The results are averaged and characterized on a scale of 0 (no itching—normal dog), 2 (very mild itching—only occasional episodes), 4 (mild itching), 6 (moderate itching—regular episodes), 8 (severe itching—prolonged episodes), to 10 (extremely severe itching—almost continuous).

Results

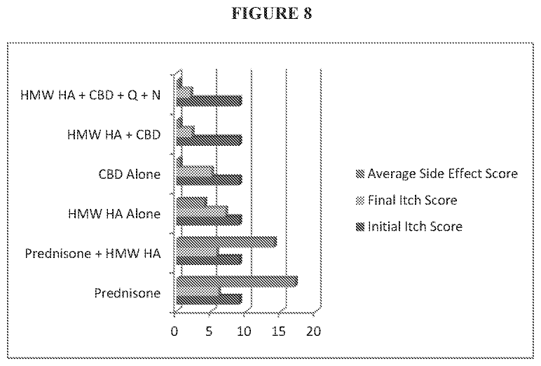

The following scores are observed and the results depicted in FIG. 8:

| | | | | Average |
|---|---|---|---|---|
| | | Initial | Final | Side |
| Group | Active | Itch | Itch | Effect |
| # | Substance(s) | Score | Score | Score |
| 1 | Prednisone | 9 | 6 | 17 |
| 2 | Prednisone + HMW HA | 9 | 5.7 | 14 |
| 3 | HMW HA Alone | 9 | 7 | 4 |
| 4 | CBD Alone | 9 | 5 | .5 |
| 5 | HMW HA + CBD | 9 | 2.2 | .5 |
| 6 | HMW HA + CBD + | 9 | 2 | .4 |
| | Quercetin + Nettles | | | |

As is evident, compositions with HMW HA and CBD as well as compositions with HMW HA, CBD, quercetin, and nettles are unexpectedly and surprisingly more effective than prednisone and prednisone and HMW HA at treating skin conditions with markedly lower side effects than prednisone and prednisone and HMW HA.

### Example 9—Pain Relief and Side Effects

This example examines the ability of various compositions to minimize pain and examines the level of adverse side effects of each composition.

Inclusion Criteria

(1) Canines over one year of age.

(2) Radiographic evidence of osteoarthritis as determined by the managing veterinarian.

(3) No other co-morbidities.

(4) Sufficient wash-out of any other nutraceutical and/or pharmaceutical and no further use of any other nutraceutical and/or pharmaceutical until completion of study.

Exclusion Criteria

Owner cannot comply with evaluation and/or follow-up requirements.

Materials and Methods

At the outset of the study, participants receive a full physical examination, including weight measurement, thigh circumference, and CBC/blood chemistries. Because osteoarthritis is not only a disease of pain, but one characterized by behavioral, appetite, anxiety/aggression, family socialization, and sleep aberrations, the pet's owners are familiarized with the pain scale (canine orthopedic index) and additional behavioral assessment log.

The patient group is twenty-four dogs suffering from osteoarthritis, broken into eight groups of three dogs. The first group receives a control composition with no active agent; the second group receives a composition comprising HA as the sole active agent; the third group receives a composition comprising HMW HA as the sole active agent; the fourth group receives a composition comprising CBD as the sole active agent; the fifth group receives a composition comprising HA and CBD as the active agents; the sixth group receives a composition comprising HMW HA and CBD Complex as the active agents; the seventh group receives a composition comprising Rimadyl as the active agent; and the eighth group receives a composition comprising Meloxicam as the active agent.

Each composition is administered for a period of four (4) weeks. The owner completes the electronic log questionnaire each week. At the end of the 4-week study, participants have another full physical examination, including weight measurement, thigh circumference, and CBC/blood chemistries, with an exit interview of the owners to gain final impressions.

Results

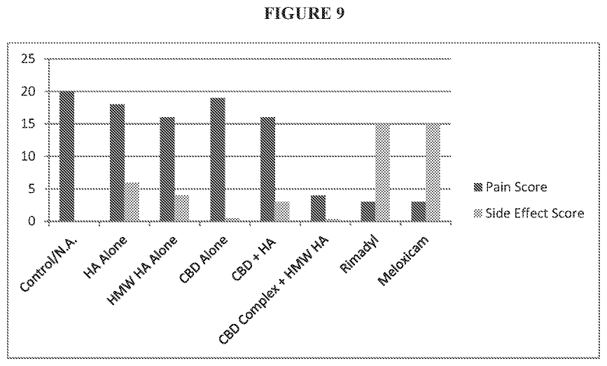

Each group is evaluated for pain scores and the scores averaged. The following scores are observed and the results depicted in FIG. 9:

| | | Average | |
|---|---|---|---|
| | | Pain | Average Side |
| Group # | Active Substance(s) | Score | Effect Score |
| 1 | Control/Not Applicable | 20 | 0 |
| 2 | HA Alone | 18 | 6 |
| 3 | HMW HA Alone | 16 | 4 |
| 4 | CBD Alone | 19 | 0.5 |
| 5 | CBD + HA | 16 | 3 |
| 6 | CBD Complex + | 4 | 3 |
| | HMW HA | | |
| 7 | Rimadyl | 3 | 15 |
| 8 | Meloxicam | 3 | 15 |

As is evident, conventional treatments for osteoarthritis of Rimadyl and Meloxicam are effective in terms of reducing pain, but bring a high degree of adverse side effects. HA alone and HMW HA alone have a low degree of adverse side effects, but only slightly reduce pain. CBD alone and CBD with HA have an even lower degree of adverse side effects than do HA alone and HMW HA alone, but are not especially effective in reducing pain. However, unexpected and surprising results are observed with the CBD Complex and HMW HA composition, which greatly reduces pain and brings a very low degree of adverse side effects.

### Example 10—Effects of HMW HA and CBD Complex Compositions on Treating Cognitive Decline

Selegiline (marketed as Anipryl®) is currently the only FDA-approved treatment for canine cognitive dysfunction. Although the onset of action can be variable, ranging from four to twelve weeks, most dogs show some improvement after one month of treatment and there may be continued treatment over time.

This example examines the ability of embodiments of the invention versus Anipryl® to treat cognitive decline.

Materials and Methods

The patient group is twenty dogs suffering from cognitive dysfunction syndrome, as determined by the Cognitive Dysfunction Screening Checklist. See G. Landsberg et al., 2003 HANDBOOK OF BEHAVIOR PROBLEMS OF THE DOG AND CAT (Elsevier Science Limited) (2003). The twenty dogs are broken into five groups of four dogs. The first group receives Anipryl® as the sole active agent; the second group receives a composition comprising HMW HA as the sole active agent; the third group receives a composition comprising CBD as the sole active agent; the fourth group receives a composition comprising HMW HA and CBD Complex as the active agents; and the fifth group receives a composition comprising HMW HA, CBD Complex, nicotinamide adenine dinucleotide (“NAD”), and phosphatidylserine as the active agents.

Each composition is administered for a period of sixty (60) days, twice per day (“BID”). The owner completes the Checklist on days 0 (“Initial Score”), 15, 30, 45, and 60 (“Final Score”) of treatment.

Results

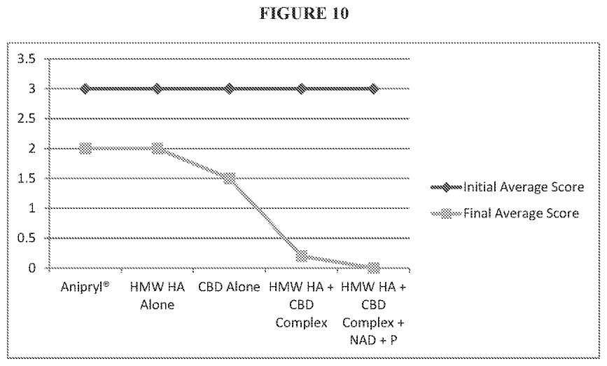

Each group is evaluated and Checklist scores averaged. The following scores are observed and the results depicted in FIG. 10:

| | | Average | Average |
|---|---|---|---|
| | | Initial | Final |
| Group # | Active Substance(s) | Score | Score |
| 1 | Anipryl ® | 3 | 2 |
| 2 | HMW HA Alone | 3 | 2 |
| 3 | CBD Alone | 3 | 1.5 |
| 4 | HMW HA + CBD Complex | 3 | .2 |
| 5 | HMW HA + CBD Complex + | 3 | 0 |
| | NAD + phosphatidylserine | | |

As is evident, Anipryl® is as effective as HMW HA alone in terms of reducing cognitive decline and CBD alone is even more effective. However, unexpected and surprising results are observed with the HMW HA+CBD Complex (which lowers the score from 3 to 0.2) and HMW HA+CBD Complex+NAD+phosphatidylserine composition (which lowers the score from 3 to 0).

### Example 11—Effects of HMW HA and CBD Complex Compositions on Treating Unwanted Gastrointestinal-Specific Inflammation

Famotidine, commonly marketed as Pepcid®, is used to help reduce the amount of stomach acid produced and can be used to treat gastritis, esophagitis, and gastric or esophageal reflux, as well as to prevent stomach or duodenal ulcers in animals with kidney failure.

This example examines the ability of embodiments of the invention versus Pepcid® to treat unwanted GI-specific inflammation characterized by gastritis, reflux, vomiting, diarrhea, and inappetence.

Materials and Methods

The first patient group is twenty dogs suffering from GI-specific inflammation. The second patient group is twenty cats suffering from GI-specific inflammation. Each patient group is broken into five groups of four animals. The first groups receive Pepcid® as the sole active agent; the second groups receive a composition comprising HMW HA as the sole active agent; the third groups receive a composition comprising CBD as the sole active agent; the fourth groups receive a composition comprising HMW HA and CBD Complex as the active agents; and the fifth groups receive a composition comprising HMW HA, CBD Complex, and glutathione as the active agents.

Each composition is administered for a period of twenty-eight (28) days, BID. The owner observes potential reduction of symptoms at the end of treatment and rates the reduction as no reduction (score of 0), mild reduction (score of 1), moderate reduction (score of 2), significant reduction (score of 3), or resolution of symptoms (score of 4).

Results

| | | Initial | Final |
|---|---|---|---|
| Group # | Active Substance(s) | Score | Score |
| 1 | Pepcid ® | 0 | 1 |
| 2 | HMW HA Alone | 0 | 3 |
| 3 | CBD Alone | 0 | 2 |
| 4 | HMW HA + CBD Complex | 0 | 4 |
| 5 | HMW HA + CBD Complex + | 0 | 4 |
| | glutathione | | |

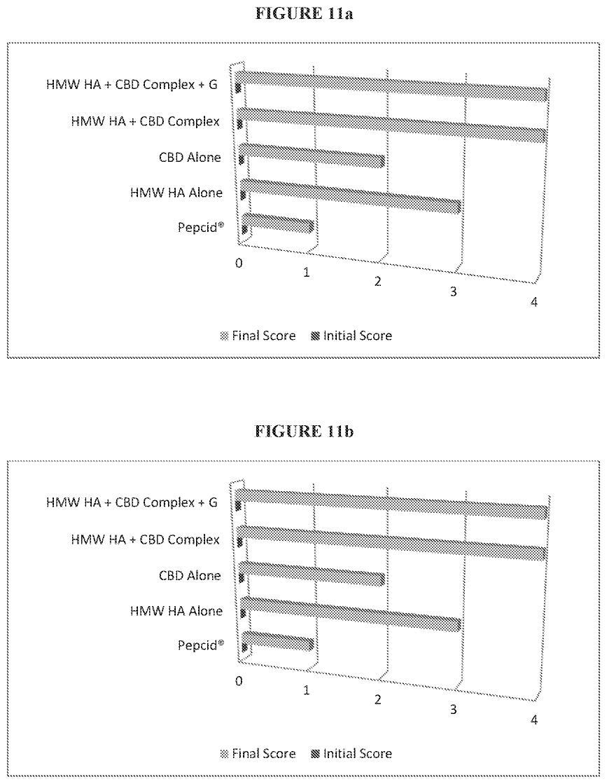

The following canine scores are observed and the results depicted in FIG. 11a:

| | | Initial | Final |
|---|---|---|---|
| Group # | Active Substance(s) | Score | Score |
| 1 | Pepcid ® | 0 | 1 |
| 2 | HMW HA Alone | 0 | 3 |
| 3 | CBD Alone | 0 | 2 |
| 4 | HMW HA + CBD Complex | 0 | 4 |
| 5 | HMW HA + CBD Complex + | 0 | 4 |
| | glutathione | | |

As is evident, compositions with HMW HA and CBD Complex as well as compositions with HMW HA, CBD Complex, and glutathione are unexpectedly and surprisingly more effective than Pepcid® at reducing symptoms of unwanted GI-specific inflammation.

### Example 12—Effects of HMW HA and CBD Complex Compositions on Treating Joint Inflammation

Cosequin®, a supplement containing TRH122®, chondroitin sulfate, FCHG49®, glucosamine, and manganese ascorbate, is a patented, scientifically-researched nutritional supplement dispensed by thousands of veterinarians to help dogs maintain healthy joints.

This example examines the ability of embodiments of the invention versus Cosequin® to treat joint inflammation.

Materials and Methods

The first patient group is twenty dogs suffering from joint inflammation. The second patient group is twenty cats suffering from joint inflammation. The third patient group is twenty horses suffering from joint inflammation. Each patient group is broken into five groups of four animals. The first groups receive Cosequin® as the sole active agent; the second groups receive a composition comprising HMW HA as the sole active agent; the third groups receive a composition comprising CBD as the sole active agent; the fourth groups receive a composition comprising HMW HA and CBD Complex as the active agents; and the fifth groups receive a composition comprising HMW HA, CBD Complex, Boswellia, and superoxide dismutase (“SOD”) as the active agents.

Each composition is administered for a period of sixty (60) days, BID. Efficacy is determined by a decrease in body temperature over the inflamed joint, as measured by thermal imaging (e.g., via Digatherm device). Thermal imaging is performed on days 0 (“Initial Score”), 15, 30, 45, and 60 (“Final Score”) of treatment.

Results

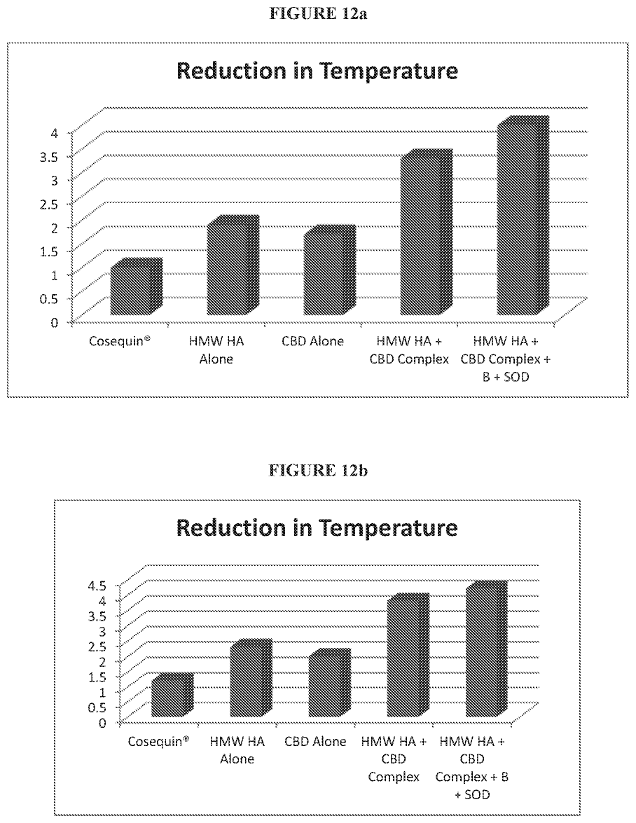

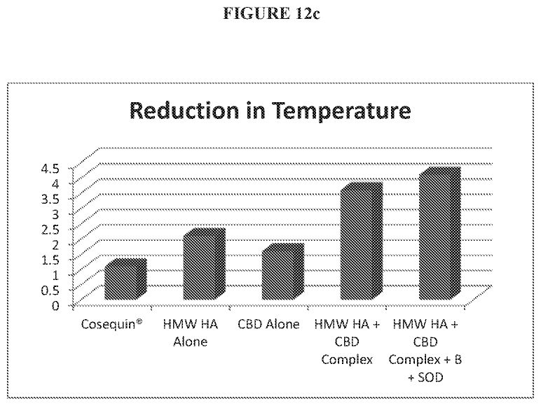

The following canine scores are observed and the results depicted in FIG. 12a:

| | | Reduction in |
|---|---|---|
| | | Temperature |
| Group # | Active Substance(s) | (° F.) |
| 1 | Cosequin ® | 1 |
| 2 | HMW HA Alone | 1.9 |
| 3 | CBD Alone | 1.7 |
| 4 | HMW HA + CBD Complex | 3.3 |
| 5 | HMW HA + CBD Complex + | 4.0 |
| | Boswellia + SOD | |

The following feline scores are observed and the results depicted in FIG. 12b:

| | | Reduction in |
|---|---|---|
| | | Temperature |
| Group # | Active Substance(s) | (° F.) |
| 1 | Cosequin ® | 1.2 |
| 2 | HMW HA Alone | 2.3 |
| 3 | CBD Alone | 2.0 |
| 4 | HMW HA + CBD Complex | 3.8 |
| 5 | HMW HA + CBD Complex + | 4.2 |
| | Boswellia + SOD | |

The following equine scores are observed and the results depicted in FIG. 12c:

| | | Reduction in |
|---|---|---|
| | | Temperature |
| Group # | Active Substance(s) | (° F.) |
| 1 | Cosequin ® | 1.1 |
| 2 | HMW HA Alone | 2.1 |
| 3 | CBD Alone | 1.6 |
| 4 | HMW HA + CBD Complex | 3.6 |
| 5 | HMW HA + CBD Complex + | 4.1 |
| | Boswellia + SOD | |

As is evident, compositions with HMW HA and CBD Complex as well as compostions with HMW HA, CBD Complex Bowsellia and SOD are unexpectedly and surprisingly more effective than Cosequin® at reducing joint inflammation.

### Example 13—Effects of HMW HA and CBD Complex Compositions on Reducing Behavior Aberrations and Anxiety

Clomicalm® is used in dogs to treat behavior problems such as separation anxiety, excessive barking, and destructive behavior. Clomicalm® is used in cats to help treat behavior problems such as urine spraying, certain types of aggression, and compulsive behaviors (such as excessive grooming).

This example examines the ability of embodiments of the invention versus Clomicalm® to reduce behavior aberrations and anxiety.

Materials and Methods

The first patient group is twenty dogs suffering behavior aberrations and/or anxiety, as determined by the canine behavioral assessment and research questionnaire (“C-BARQ”). See, e.g., P. Wiener et al., Use of questionnaire-based data to assess dog personality, 16 JOURNAL OF VETERINARY BEHAVIOR 81-85 (2016). The second patient group is twenty cats suffering behavior aberrations and/or anxiety, as determined by the feline behavioral assessment and research questionnaire (“Fe-BARQ”).

Each group is broken into five groups of four animals. The first group receives Clomicalm® as the sole active agent; the second group receives a composition comprising HMW HA as the sole active agent; the third group receives a composition comprising CBD as the sole active agent; the fourth group receives a composition comprising HMW HA and CBD Complex as the active agents; and the fifth group receives a composition comprising HMW HA, CBD Complex, valerian root, and scutillaria as the active agents.

Each composition is administered for a period of sixty (60) days, once a day (“SID”). Owners complete the species-appropriate C-BARQ or Fe-BARQ on days 0 and 60 of treatment. The results from the overall questionnaire are averaged and characterized as no improvement (score of 0), mild improvement (score of 1), moderate improvement (score of 2), or marked improvement (score of 3).

Results

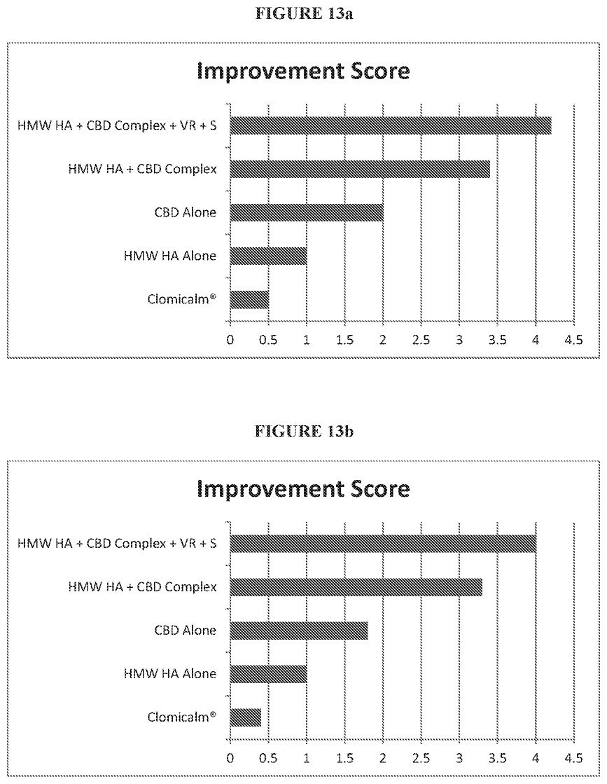

The following canine scores are observed and the results depicted in FIG. 13a:

| | | Improvement |
|---|---|---|
| Group # | Active Substance(s) | Score |
| 1 | Clomicalm ® | .5 |
| 2 | HMW HA Alone | 1 |
| 3 | CBD Alone | 2 |
| 4 | HMW HA + CBD Complex | 3.4 |
| 5 | HMW HA + CBD Complex + | 4.2 |
| | Valerian Root + Scutillaria | |

The following feline scores are observed and the results depicted in FIG. 13b:

| | | Improvement |
|---|---|---|
| Group # | Active Substance(s) | Score |
| 1 | Clomicalm ® | .4 |
| 2 | HMW HA Alone | 1 |
| 3 | CBD Alone | 1.8 |
| 4 | HMW HA + CBD Complex | 3.3 |
| 5 | HMW HA + CBD Complex + | 4.0 |
| | Valerian Root + Scutillaria | |

As is evident, compositions with HMW HA and CBD Complex as well as compositions with HMW HA, CBD Complex, valerian root, and scutillaria are unexpectedly and surprisingly more effective than Clomicalm® at reducing behavior aberrations and anxiety.

### Example 14—Effects of HMW HA and CBD Complex Compositions on Treating Seizures

Phenobarbital, while not FDA-approved for use in dogs, is the primary medication used to treat seizures in dogs. Phenobarbital is inexpensive, easy to use, and augments the activity of GABA in the brain, which stops signals from passing from one neuron to the next. Taking phenobarbital daily stops seizures in dogs by reducing electrical energy. Understandably, many side effects of phenobarbital are neurological, making the dog lethargic, sedated, restless, hyper-excited, and/or uncoordinated. These side effects may or may not resolve after several weeks if the dog's system becomes used to the medication. Long-term side effects of phenobarbital include frequent urination, excessive drinking, and excessive eating (which, in turn, can result in weight gain). The most dangerous side effect of phenobarbital is liver damage. While it occurs in a small percentage of dogs, long-term use of phenobarbital can case scarring of the liver and subsequent liver failure.

This example examines the ability of embodiments of the invention versus phenobarbital to treat seizures.

Materials and Methods

The patient group is twenty-four dogs suffering from seizures as diagnosed by their referring veterinarian. The patient group is broken into six groups of four animals. The first group receives phenobarbital as the sole active agent; the second group receives a composition comprising phenobarbital and Keppra®; the third group receives a composition comprising HMW HA as the sole active agent; the fourth group receives a composition comprising CBD as the sole active agent; the fifth group receives a composition comprising HMW HA and CBD Complex as the active agents; and the sixth group receives a composition comprising HMW HA, CBD Complex, valerian root, and scutillaria as the active agents.

Each composition is administered for a period of sixty (60) days, BID. Each dog is evaluated on days 0 (“Initial Score”), 15, 30, 45, and 60 (“Final Score”) of treatment. The results are averaged and characterized on a scale of 1 (seizure-free) to 10 (daily seizures or multiple seizures during the event).

Results

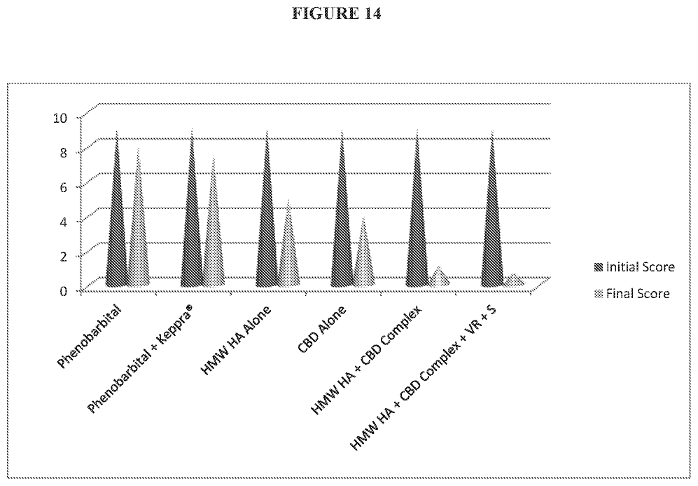

The following canine scores are observed and the results depicted in FIG. 14:

| | | Initial | Final |
|---|---|---|---|
| Group # | Active Substance(s) | Score | Score |
| 1 | Phenobarbital | 9 | 8 |
| 2 | Phenobarbital + Keppra ® | 9 | 7.5 |
| 3 | HMW HA Alone | 9 | 5 |
| 4 | CBD Alone | 9 | 4 |
| 5 | HMW HA + CBD Complex | 9 | 1.1 |
| 6 | HMW HA + CBD Complex + | 9 | 0.7 |
| | Valerian Root + Scutillaria | | |

As is evident, compositions with HMW HA and CBD Complex as well as compositions with HMW HA, CBD Complex, valerian root, and scutillaria are unexpectedly and surprisingly more effective than phenobarbital and phenobarbital and Keppra® at reducing seizures.

### Example 15—Effects of HMW HA and CBD Complex Compositions on Cardiac Function

Enalapril is an angiotensin-converting enzyme (“ACE”) inhibitor that is commonly prescribed by veterinarians for the treatment of cardiac issues in dogs. Enalapril relaxes blood vessels, which allows blood to flow more freely, reduce blood pressure, and reduce the heart's workload. Enalapril is useful for treating heart failure, heart murmurs, and high blood pressure. Side effects with enalapril include allergic reaction (resulting in hives and/or labored breathing), vomiting, diarrhea, decreased appetite, dizziness, fainting, digestive tract ulcers, high potassium levels, fever, and lethargy.

This example examines the ability of embodiments of the invention versus enalapril to treat heart failure.

Materials and Methods

The patient group is twenty-four dogs suffering from heart failure.

The patient group is broken into six groups of four dogs. The first group receives enalapril as the sole active agent; the second group receives a composition comprising enalapril and Lasix®; the third group receives a composition comprising HMW HA as the sole active agent; the fourth group receives a composition comprising CBD as the sole active agent; the fifth group receives a composition comprising HMW HA and CBD Complex as the active agents; and the sixth group receives a composition comprising HMW HA, CBD Complex, liposomal CoQ10, hawthorn berry, and ginko biloba as the active agents.

Each composition is administered for a period of ninety (90) days, BID. Each animal is evaluated on days 0 (“Initial Score”), 30, 60, and 90 (“Final Score”) of treatment. The results are averaged and characterized on a scale of 1 (few to no clinical signs of heart failure) to 10 (severe heart failure).

Results

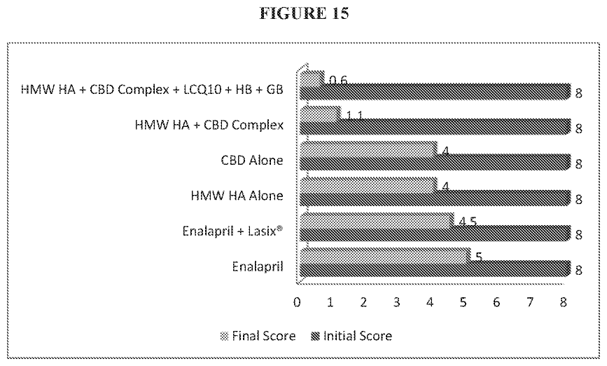

The following canine scores are observed and the results depicted in FIG. 15:

| | | Initial | Final |
|---|---|---|---|
| Group # | Active Substance(s) | Score | Score |
| 1 | Enalapril | 8 | 5 |
| 2 | Enalapril + Lasix ® | 8 | 4.5 |
| 3 | HMW HA Alone | 8 | 4 |
| 4 | CBD Alone | 8 | 4 |
| 5 | HMW HA + CBD Complex | 8 | 1.1 |
| 6 | HMW HA + CBD Complex + | 8 | 0.6 |
| | liposomal CoQ10 + hawthorn | | |
| | berry + ginko biloba | | |

As is evident, compositions with HMW HA and CBD Complex as well as compositions with HMW HA, CBD Complex, liposomal CoQ10, hawthorn berry, and ginko biloba are unexpectedly and surprisingly more effective than enalapril and enalapril and Lasix® at treating heart failure.

### Example 16—Effects of HMW HA and CBD Complex Compositions on Skin Conditions

Prednisone is a corticosteroid commonly used to treat dogs who suffer from skin allergies and autoimmune-based skin diseases. Side effects of prednisone include increased thirst and hunger, panting, loss of energy, vomiting, and/or skin infections. Long-term side effects of prednisone use include obesity (due to increased hunger), inability to heal fully from infection, urinary tract infections, development of hard calcium deposits on the skin, muscle weakness, distended stomach, and diabetes. Liver damage and iatrogenic Cushing's disease are the most serious side effects of prednisone use.

This example examines the ability of embodiments of the invention versus prednisone to treat skin conditions.

Materials and Methods

The patient group is twenty-four dogs with a history of severe, chronic skin issues, atopy, pruritis, yeast overgrowth, and/or infection, as diagnosed by their referring veterinarian. The patient group is broken into six groups of four animals. The first group receives prednisone as the sole active agent; the second group receives a composition comprising prednisone and HMW HA as the active agents; the third group receives a composition comprising HMW HA as the sole active agent; the fourth group receives a composition comprising CBD as the sole active agent; the fifth group receives a composition comprising HMW HA and CBD Complex as the active agents; and the sixth group receives a composition comprising HMW HA, CBD Complex, quercetin, and nettles as the active agents.

Each composition is administered for a period of sixty (60) days, BID. Each dog is evaluated on days 0 (“Initial Score”), 20, 40, and 60 (“Final Score”) of treatment. The results are averaged and characterized on a scale of 0 (no itching—normal dog), 2 (very mild itching—only occasional episodes), 4 (mild itching), 6 (moderate itching—regular episodes), 8 (severe itching—prolonged episodes), to 10 (extremely severe itching—almost continuous).

Results

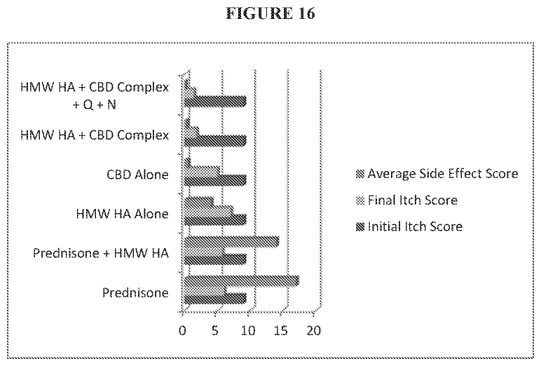

The following scores are observed and the results depicted in FIG. 16:

| | | | | Average |
|---|---|---|---|---|
| | | Initial | Final | Side |
| | Active | Itch | Itch | Effect |
| Group # | Substance(s) | Score | Score | Score |
| 1 | Prednisone | 9 | 6 | 17 |
| 2 | Prednisone + HMW HA | 9 | 5.7 | 14 |
| 3 | HMW HA Alone | 9 | 7 | 4 |
| 4 | CBD Alone | 9 | 5 | .5 |
| 5 | HMW HA + CBD Complex | 9 | 1.8 | .3 |
| 6 | HMW HA + CBD Complex + | 9 | 1.4 | .1 |
| | Quercetin + Nettles | | | |

As is evident, compositions with HMW HA and CBD Complex as well as compositions with HMW HA, CBD Complex, quercetin, and nettles are unexpectedly and surprisingly more effective than prednisone and prednisone and HMW HA at treating skin conditions with markedly lower side effects than prednisone and prednisone and HMW HA.

What is claimed is:

1. A composition comprising:
(a) hyaluronic acid; and
(b) cannabidiol
wherein said hyaluronic acid has a molecular weight of at least 800,000 Daltons.

2. A composition comprising:
(a) about 0.2% to about 10.0% hyaluronic acid by weight of the total composition; and
(b) about 0.3% to about 45% cannabidiol by weight of the total composition,
wherein said hyaluronic acid has a molecular weight of at least 800,000 Daltons.

3. The composition according to claim 1 or claim 2, wherein said cannabidiol is present at about 0.5% to about 25.0% of the total weight of the composition.

4. The composition according to any one of claims 1-3, wherein said cannabidiol is present at about 0.5% to about 15.0% of the total weight of the composition.

5. The composition according to any of claims 1-4, wherein said cannabidiol is present at about 8.0% of the total weight of the composition.

6. The composition according to any one of claims 1-5, wherein said hyaluronic acid has a molecular weight of at least 900,000 Daltons.

7. The composition according to any one of claims 1-6, wherein said hyaluronic acid has a molecular weight of at least 1,000,000 Daltons.

8. The composition according to any one of claims 1-7, wherein said hyaluronic acid has a molecular weight of at least 1,100,000 Daltons.

9. The composition according to any one of claims 1-8, further comprising at least one additional active agent.

10. The composition according to claim 9, wherein said additional active agent is Boswellia, catalase, ginko biloba, glutathione, gotu kola, hawthorn berry, kava, liposomal CoQ10, lutein, nettles, nicotinamide adenine dinucleotide, phosphatidylserine, quercetin, scutillaria, superoxide dismutase (“SOD”), valerian root, or zeaxanthin.

11. The composition according to any one of claims 1-10, further comprising a veterinarily-acceptable preservative.

12. The composition according to any one of claims 1-11, further comprising a veterinarily-acceptable flavorant.

13. The composition according to any one of claims 1-12, further comprising a veterinarily-acceptable adjuvant, carrier, and/or vehicle.

14. A method for alleviating at least one ailment and/or symptoms of at least one ailment in a non-human subject, comprising administering to said non-human subject an effective amount of the composition according to any one of claims 1-13.

15. The method according to claim 14, wherein said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian.

16. The method according to claim 15, wherein said non-human subject is a bird, cat, dog, elephant, ferret, guinea pig, hamster, hedgehog, horse, kangaroo, llama, monkey, mouse, otter, pig, rabbit, rat, seal, or weasel.

17. The method according to any one of claims 14-16, wherein said administration is by inhalation, intramuscular, intranasal, intravenous, oral, rectal, subcutaneous, sublingual, transdermal, transmucosal, or combinations thereof.

18. The method according to any one of claims 14-17, wherein said composition is provided in aerosol, capsule, cream, dispersion, gel, gelatin capsule, granule, liquid, lotion, lozenge, ointment, paste, patch, pill, powder, solution, spray, suppository, suspension, syrup, or tablet (coated or uncoated) form.

19. The method according to any one of claims 14-18, wherein said ailment is arthritis, behavioral disorder, cancer, cardiovascular disorder, cognitive decline, dermatitis, gastrointestinal disorder, inflammation, irritability, nausea, neurological disorder, osteoarthritis, pain, poor appetite, pruritis, seizure, or skin disorder.

20. The method according to claim 19, wherein said behavioral disorder is aggression, general anxiety, noise phobia, separation anxiety, and/or sleep disturbance.

21. The method according to claim 19, wherein said osteoarthritis manifests as ataxia, gait deficit, pain, stiffness, and/or weakness.

22. The method according to claim 19, wherein said neurologic disorder manifests as ataxia, disorientation, lameness, pain, paralysis, paresis, seizure, and/or tremors.

23. The method according to claim 19, wherein said pain manifests as aggression, decreased appetite, gait deficits, inappropriate eliminations, increased sleeping, lethargy, night restlessness, panting, self segregation, sleep disturbance, and/or vocalizations.

24. The method according to claim 19, wherein said cognitive decline manifests as apathy, confusion, house soiling, impaired ability for commands, impaired ability for tasks, impaired ability for work, increased anxiety, increased irritability, negative changes in activity, negative changes in learning, negative changes in memory, negative changes in relationships, negative changes in sleep-wake cycles, negative changes in social behavior, problems with awareness, problems with spatial orientation, and/or repetitive activity.

25. The method according to claim 19, wherein said gastrointestinal disorder manifests as anorexia, bloating, constipation, belching, diarrhea, gas, gastroesophageal reflex disease (“GERD”) and symptoms thereof, hiccups, and/or vomiting.

26. The method according to any one of claims 14-25, wherein about 0.05 mg/kg to about 20.0 mg/kg of cannabidiol by weight of said subject is administered; preferably about 0.25 mg/kg to about 12.0 mg/kg; more preferably about 0.50 mg/kg to about 10.0 mg/kg; and even more preferably about 1.0 mg/kg to about 10.0 mg/kg.

27. The method according to any one of claims 14-25, wherein about 5.0 mg/kg to about 10.0 mg/kg of cannabidiol by weight of said subject is administered.

28. The method according to any one of claims 14-25, wherein about 8.0 mg/kg of cannabidiol by weight of said subject is administered.

29. The method according to any one of claims 14-28, wherein said composition is administered twice a day for one week and then once a day thereafter.

30. The method according to any one of claims 14-28, wherein said composition is administered at least once a day.

31. The method according to any one of claims 14-30, wherein said composition is administered orally.

32. A method for treating cognitive decline in a non-human subject, said method comprising administering to said non-human subject in need thereof an effective amount of a composition according to any one of claims 1-13, wherein said at least one additional active agent is glutathione, ginko biloba, gotu kola, nicotinamide adenine dinucleotide, and/or phosphatidylserine.

33. A method for treating unwanted inflammation in a non-human subject, said method comprising administering to said non-human subject in need thereof an effective amount of a composition according to any one of claims 1-13, wherein said at least one additional active agent is glutathione, ginko biloba, and/or gotu kola.

34. A method for supporting oxidative stress and/or cell metabolism in a non-human subject, said method comprising administering to said non-human subject in need thereof an effective amount of a composition according to any one of claims 1-13, wherein said at least one additional active agent is catalase and/or CoQ10.

35. A method for providing joint support to and/or treating unwanted inflammation related to osteoarthritis in a non-human subject, said method comprising administering to said non-human subject in need thereof an effective amount of a composition according to any one of claims 1-13, wherein said at least one additional active agent is Boswellia and/or superoxide dismutase.

36. A method for treating anxiety and/or behavior disorder and/or sleep disturbance in a non-human subject, said method comprising administering to said non-human subject in need thereof an effective amount of a composition according to any one of claims 1-13, wherein said at least one additional active agent is kava, scutillaria, and/or valerian root.

37. A method for treating seizures in a non-human subject, said method comprising administering to said non-human subject in need thereof an effective amount of a composition according to any one of claims 1-13, wherein said at least one additional active agent is valerian root and/or scutillaria.

38. A method for providing cardiovascular support to a non-human subject, said method comprising administering to said non-human subject in need thereof an effective amount of a composition according to any one of claims 1-13, wherein said at least one additional active agent is liposomal CoQ10, hawthorn berry, and/or ginko biloba.

39. A method for treating at least one acute and/or chronic pruritic skin condition and/or at least one inflammatory skin condition of a non-human subject, said method comprising administering to said non-human subject in need thereof an effective amount of a composition according to any one of claims 1-13, wherein said at least one additional active agent is quercetin and/or nettles.

40. A method for treating declining vision in a non-human subject, said method comprising administering to said non-human subject in need thereof an effective amount of a composition according to any one of claims 1-13, wherein said at least one additional active agent is lutein and/or zeaxanthin.

41. The method according to any one of claims 32-40, wherein said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian.

42. The method according to claim 41, wherein said non-human subject is a bird, cat, dog, elephant, ferret, guinea pig, hamster, hedgehog, horse, kangaroo, llama, monkey, mouse, otter, pig, rabbit, rat, seal, or weasel.

43. The method according to any one of claims 32-42, wherein said administration is by inhalation, intramuscular, intranasal, intravenous, oral, rectal, subcutaneous, sublingual, transdermal, transmucosal, or combinations thereof.

44. The method according to any one of claims 32-42, wherein said composition is provided in aerosol, capsule, cream, dispersion, gel, gelatin capsule, granule, liquid, lotion, lozenge, ointment, paste, patch, pill, powder, solution, spray, suppository, suspension, syrup, or tablet (coated or uncoated) form.

45. The method according to any one of claims 32-44, wherein about 0.05 mg/kg to about 20.0 mg/kg of cannabidiol by weight of said subject is administered; preferably about 0.25 mg/kg to about 12.0 mg/kg; more preferably about 0.50 mg/kg to about 10.0 mg/kg; and even more preferably about 1.0 mg/kg to about 10.0 mg/kg.

46. The method according to any one of claims 32-44, wherein about 5.0 mg/kg to about 10.0 mg/kg of cannabidiol by weight of said subject is administered.

47. The method according to any one of claims 32-44, wherein about 8.0 mg/kg of cannabidiol by weight of said subject is administered.

48. The method according to any one of claims 32-47, wherein said composition is administered twice a day for one week and then once a day thereafter.

49. The method according to any one of claims 32-47, wherein said composition is administered at least once a day.

50. The method according to any one of claims 32-49, wherein said composition is administered orally.

51. A composition comprising:
(a) hyaluronic acid; and
(b) cannabidiol (“CBD”) Complex
wherein said hyaluronic acid has a molecular weight of at least 800,000 Daltons.

52. A composition comprising:
(a) about 0.2% to about 10.0% hyaluronic acid by weight of the total composition; and
(b) cannabidiol (“CBD”) Complex,
wherein said hyaluronic acid has a molecular weight of at least 800,000 Daltons and wherein CBD is present at about 0.3% to about 45% by weight of the total composition.

53. The composition according to claim 51 or claim 52, wherein said cannabidiol is present at about 0.5% to about 25.0% of the total weight of the composition.

54. The composition according to any one of claims 51-53, wherein said cannabidiol is present at about 0.5% to about 15.0% of the total weight of the composition.

55. The composition according to any of claims 51-54, wherein said cannabidiol is present at about 8.0% of the total weight of the composition.

56. The composition according to any one of claims 51-55, wherein said hyaluronic acid has a molecular weight of at least 900,000 Daltons.

57. The composition according to any one of claims 51-56, wherein said hyaluronic acid has a molecular weight of at least 1,000,000 Daltons.

58. The composition according to any one of claims 51-57, wherein said hyaluronic acid has a molecular weight of at least 1,100,000 Daltons.

59. The composition according to any one of claims 51-58, further comprising at least one additional active agent.

60. The composition according to claim 59, wherein said additional active agent is Boswellia, catalase, ginko biloba, glutathione, gotu kola, hawthorn berry, kava, liposomal CoQ10, lutein, nettles, nicotinamide adenine dinucleotide, phosphatidylserine, quercetin, scutillaria, superoxide dismutase (“SOD”), valerian root, or zeaxanthin.

61. The composition according to any one of claims 51-60, further comprising a veterinarily-acceptable preservative.

62. The composition according to any one of claims 51-61, further comprising a veterinarily-acceptable flavorant.

63. The composition according to any one of claims 51-62, further comprising a veterinarily-acceptable adjuvant, carrier, and/or vehicle.

64. A method for alleviating at least one ailment and/or symptoms of at least one ailment in a non-human subject, comprising administering to said non-human subject an effective amount of the composition according to any one of claims 51-63.

65. The method according to claim 64, wherein said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian.

66. The method according to claim 65, wherein said non-human subject is a bird, cat, dog, elephant, ferret, guinea pig, hamster, hedgehog, horse, kangaroo, llama, monkey, mouse, otter, pig, rabbit, rat, seal, or weasel.

67. The method according to any one of claims 64-66, wherein said administration is by inhalation, intramuscular, intranasal, intravenous, oral, rectal, subcutaneous, sublingual, transdermal, transmucosal, or combinations thereof.

68. The method according to any one of claims 64-67, wherein said composition is provided in aerosol, capsule, cream, dispersion, gel, gelatin capsule, granule, liquid, lotion, lozenge, ointment, paste, patch, pill, powder, solution, spray, suppository, suspension, syrup, or tablet (coated or uncoated) form.

69. The method according to any one of claims 64-68, wherein said ailment is arthritis, behavioral disorder, cancer, cardiovascular disorder, cognitive decline, dermatitis, gastrointestinal disorder, inflammation, irritability, nausea, neurological disorder, osteoarthritis, pain, poor appetite, pruritis, seizure, or skin disorder.

70. The method according to claim 69, wherein said behavioral disorder is aggression, general anxiety, noise phobia, separation anxiety, and/or sleep disturbance.

71. The method according to claim 69, wherein said osteoarthritis manifests as ataxia, gait deficit, pain, stiffness, and/or weakness.

72. The method according to claim 69, wherein said neurologic disorder manifests as ataxia, disorientation, lameness, pain, paralysis, paresis, seizure, and/or tremors.

73. The method according to claim 69, wherein said pain manifests as aggression, decreased appetite, gait deficits, inappropriate eliminations, increased sleeping, lethargy, night restlessness, panting, self segregation, sleep disturbance, and/or vocalizations.

74. The method according to claim 69, wherein said cognitive decline manifests as apathy, confusion, house soiling, impaired ability for commands, impaired ability for tasks, impaired ability for work, increased anxiety, increased irritability, negative changes in activity, negative changes in learning, negative changes in memory, negative changes in relationships, negative changes in sleep-wake cycles, negative changes in social behavior, problems with awareness, problems with spatial orientation, and/or repetitive activity.

75. The method according to claim 69, wherein said gastrointestinal disorder manifests as anorexia, bloating, constipation, belching, diarrhea, gas, gastroesophageal reflex disease (“GERD”) and symptoms thereof, hiccups, and/or vomiting.

76. The method according to any one of claims 64-75, wherein about 0.05 mg/kg to about 20.0 mg/kg of cannabidiol by weight of said subject is administered; preferably about 0.25 mg/kg to about 12.0 mg/kg; more preferably about 0.50 mg/kg to about 10.0 mg/kg; and even more preferably about 1.0 mg/kg to about 10.0 mg/kg.

77. The method according to any one of claims 64-75, wherein about 5.0 mg/kg to about 10.0 mg/kg of cannabidiol by weight of said subject is administered.

78. The method according to any one of claims 64-75, wherein about 8.0 mg/kg of cannabidiol by weight of said subject is administered.

79. The method according to any one of claims 64-78, wherein said composition is administered twice a day for one week and then once a day thereafter.

80. The method according to any one of claims 64-78, wherein said composition is administered at least once a day.

81. The method according to any one of claims 64-80, wherein said composition is administered orally.

82. A method for treating cognitive decline in a non-human subject, said method comprising administering to said non-human subject in need thereof an effective amount of a composition according to any one of claims 51-63, wherein said at least one additional active agent is glutathione, ginko biloba, gotu kola, nicotinamide adenine dinucleotide, and/or phosphatidylserine.

83. A method for treating unwanted inflammation in a non-human subject, said method comprising administering to said non-human subject in need thereof an effective amount of a composition according to any one of claims 51-63, wherein said at least one additional active agent is glutathione, ginko biloba, and/or gotu kola.

84. A method for supporting oxidative stress and/or cell metabolism in a non-human subject, said method comprising administering to said non-human subject in need thereof an effective amount of a composition according to any one of claims 51-63, wherein said at least one additional active agent is catalase and/or CoQ10.

85. A method for providing joint support to and/or treating unwanted inflammation related to osteoarthritis in a non-human subject, said method comprising administering to said non-human subject in need thereof an effective amount of a composition according to any one of claims 51-63, wherein said at least one additional active agent is Boswellia and/or superoxide dismutase.

86. A method for treating anxiety and/or behavior disorder and/or sleep disturbance in a non-human subject, said method comprising administering to said non-human subject in need thereof an effective amount of a composition according to any one of claims 51-63, wherein said at least one additional active agent is kava, scutillaria, and/or valerian root.

87. A method for treating seizures in a non-human subject, said method comprising administering to said non-human subject in need thereof an effective amount of a composition according to any one of claims 51-63, wherein said at least one additional active agent is valerian root and/or scutillaria.

88. A method for providing cardiovascular support to a non-human subject, said method comprising administering to said non-human subject in need thereof an effective amount of a composition according to any one of claims 51-63, wherein said at least one additional active agent is liposomal CoQ10, hawthorn berry, and/or ginko biloba.

89. A method for treating at least one acute and/or chronic pruritic skin condition and/or at least one inflammatory skin condition of a non-human subject, said method comprising administering to said non-human subject in need thereof an effective amount of a composition according to any one of claims 51-63, wherein said at least one additional active agent is quercetin and/or nettles.

90. A method for treating declining vision in a non-human subject, said method comprising administering to said non-human subject in need thereof an effective amount of a composition according to any one of claims 51-63, wherein said at least one additional active agent is lutein and/or zeaxanthin.

91. The method according to any one of claims 82-90, wherein said non-human subject is avine, canine, cercopithecine, cricetine, elephantine, equine, feline, glirine, lapine, leporine, macropine, murine, musteline, non-human primate, otarine, ovine, porcine, psittacine, rodentine, or simian.

92. The method according to claim 91, wherein said non-human subject is a bird, cat, dog, elephant, ferret, guinea pig, hamster, hedgehog, horse, kangaroo, llama, monkey, mouse, otter, pig, rabbit, rat, seal, or weasel.

93. The method according to any one of claims 82-92, wherein said administration is by inhalation, intramuscular, intranasal, intravenous, oral, rectal, subcutaneous, sublingual, transdermal, transmucosal, or combinations thereof.

94. The method according to any one of claims 82-92, wherein said composition is provided in aerosol, capsule, cream, dispersion, gel, gelatin capsule, granule, liquid, lotion, lozenge, ointment, paste, patch, pill, powder, solution, spray, suppository, suspension, syrup, or tablet (coated or uncoated) form.

95. The method according to any one of claims 82-94, wherein about 0.05 mg/kg to about 20.0 mg/kg of cannabidiol by weight of said subject is administered; preferably about 0.25 mg/kg to about 12.0 mg/kg; more preferably about 0.50 mg/kg to about 10.0 mg/kg; and even more preferably about 1.0 mg/kg to about 10.0 mg/kg.

96. The method according to any one of claims 82-94, wherein about 5.0 mg/kg to about 10.0 mg/kg of cannabidiol by weight of said subject is administered.

97. The method according to any one of claims 82-94, wherein about 8.0 mg/kg of cannabidiol by weight of said subject is administered.

98. The method according to any one of claims 82-97, wherein said composition is administered twice a day for one week and then once a day thereafter.

99. The method according to any one of claims 82-97, wherein said composition is administered at least once a day.

100. The method according to any one of claims 82-99, wherein said composition is administered orally.
